(19) United States
(12) Patent Application Publication
(10) Pub. No.: US 2026/0198867 A1
(43) Pub. Date: Jul. 16, 2026

(54) GLUCOSE LEVEL INDICATOR MODEL WITH MODIFIED ERROR RATE

(71) Applicant: ARIZONA BOARD OF REGENTS ON BEHALF OF ARIZONA STATE UNIVERSITY, Scottsdale, AZ (US)

(72) Inventors: Hassan Zadeh, Phoenix, AZ (US); Saman Khamesian, Tempe, AZ (US)

(21) Appl. No.: 19/438,256

(22) Filed: Dec. 31, 2025

Related U.S. Application Data
(60) Provisional application No. 63/740,772, filed on Dec. 31, 2024.

(51) Int. Cl. A61B 5/00; A61B 5/145

(52) U.S. Cl. CPC A61B 5/7275; A61B 5/0004; A61B 5/14532; A61B 5/7267

(57) ABSTRACT

A system is described herein comprising at least one application running on one or more processors of a server, wherein the at least one application is communicatively coupled with one or more sensor devices and a remote mobile device, the one or more sensor devices configured to monitor real time biometric data of a subject, the one or more sensing devices configured to transmit the real time biometric data to the at least one application, the at least one application configured to extract feature data variables from the real time biometric data, apply a predictive model to the feature data variables to predict glucose levels of the subject over a future period of time, and communicate a signal to the mobile device when predicted glucose levels cross either an upper or lower threshold.

### RELATED APPLICATIONS

This application claims priority to U.S. Application No. 63/740,772, filed Dec. 31, 2024.

### GOVERNMENT INTERESTS

This invention was made with government support under 2402650 awarded by the National Science Foundation. The government has certain rights in the invention.

### TECHNICAL FIELD

The disclosure herein involves systems and methods for predicting glucose levels.

### BACKGROUND

TYPE 1 diabetes (TID) is an autoimmune condition characterized by the destruction of insulin-producing beta cells in the pancreas, leading to a lifelong dependency on exogenous insulin. Managing TID is particularly challenging due to the need for continuous monitoring and precise insulin dosing to maintain blood glucose levels within a target range. Poor glucose control can lead to severe complications, including cardiovascular diseases, neuropathy, retinopathy, and kidney failure [1]-[3]. Effective management is crucial for enhancing quality of life and reducing the long-term complications associated with TID. It is estimated that around 8.4 million people worldwide have TID, representing approximately 5-10% of all diabetes cases [4]. The prevalence of TID varies significantly across different populations and regions, highlighting the importance of tailored management strategies to address the unique needs of patients globally. Over the years, several advanced technologies have emerged to aid in managing TID [4]. These technologies aim to improve glycemic control, reduce the risk of complications, and alleviate the daily burden of diabetes management on patients. However, improved methods of glucose monitoring are needed.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A system is described herein comprising under an embodiment at least one application running on one or more processors of a server, wherein the at least one application is communicatively coupled with one or more sensor devices and a remote mobile device, the one or more sensor devices configured to monitor real time biometric data of a subject, the one or more sensing devices configured to transmit the real time biometric data to the at least one application, the at least one application configured to extract feature data variables from the real time biometric data, apply a predictive model to the feature data variables to predict glucose levels of the subject over a future period of time, and communicate a signal to the mobile device when predicted glucose levels cross either an upper or lower threshold.

In embodiments, the one or more sensor devices comprises a t: Slim X2™ insulin pump.

In embodiments, the one or more devices comprises a Dexcom G6™ interstitial glucose sensor.

In embodiments, the feature data variables comprise an amount of basal insulin delivered to the subject every hour.

In embodiments, the feature data variables comprise an amount of carbohydrates in grams consumed by the subject.

In embodiments, the feature data variables comprise an amount of bolus insulin scheduled for release.

In embodiments, the feature data variables comprise raw continuous glucose levels.

In embodiments, the feature data variables comprise moving average glucose levels.

In embodiments, the feature data variables comprise glucose level classifications.

In embodiments, a first glucose level classification comprises a glucose level below a first threshold.

In embodiments, a second glucose level classification comprises a glucose level equal to or above the first threshold and equal to or below a second threshold.

In embodiments, a third glucose level classification comprises a glucose level above the second threshold.

In embodiments, the predictive model comprises a CNN-LSTM model, wherein a loss function of the CNN-LSTM model comprises a mean square error function, wherein the mean square errors are weighted according to the glucose level classifications, wherein output of the mean square error function comprises a summation of weighted errors.

In embodiments, the predictive model optimizes the weights using training data collected from a population of subjects, wherein the optimizing comprises identifying weights that minimize each subject's loss function.

In embodiments, the optimizing comprises for each subject randomly selecting a subset of candidates from the population.

In embodiments, the optimizing comprises for each candidate setting a second weight corresponding to the second glucose level classification to one.

In embodiments, the optimizing comprises for each candidate setting a first weight and a third weight corresponding to the first glucose level classification and the third glucose level classification to values within a range.

In embodiments, the optimizing comprises for each candidate deriving a fitness value by applying the predictive model to the corresponding training data of the subset of candidates, wherein the fitness value comprises output of the loss function.

In embodiments, the optimizing comprises adjusting the first weight and the third weight of candidates and candidate offspring in the subset using a genetic algorithm and information of candidate and candidate offspring fitness values

In embodiments, the optimizing comprises for each subject iteratively applying the greedy algorithm across a plurality of generations to identify optimum weights for the subject.

### BRIEF DESCRIPTION OF THE DRAWINGS

FIG. 1 shows application of a Glucose Level Indicator Model with Modified Error Rate (GLIMMER) model in a type 1 diabetes (TID) management system integrated with sensor technology, under an embodiment.

FIG. 2 shows a block diagram of a multiple linear regression model, under an embodiment.

FIG. 3 shows continuous glucose monitoring readings that highlight the regions and thresholds, under an embodiment.

FIG. 4 shows the proposed methodology of GLIMMER in predicting blood glucose levels in patients, under an embodiment.

FIG. 5 shows processing of time-series data for use in training a Convolutional Long Short-Term Memory (CNN-LSTM) model, under an embodiment.

FIG. 6 shows a diagram of the proposed CNN-LSTM architecture, under an embodiment.

FIG. 7 shows the best pair of weights for all patients in OhioT1DM dataset and the average point, under an embodiment.

FIGS. 8A-8D shows Clarke Error Grid data, under an embodiment.

FIG. 9 shows a forecasting comparison between GLIMMER and the basic CNN-LSTM model, under an embodiment.

FIG. 10 shows glucose monitoring readings that highlight the regions and thresholds, under an embodiment.

FIG. 11 shows a proposed methodology of GLIMMER in predicting blood glucose levels in patients, under an embodiment.

FIG. 12 shows a forecasting comparison between GLIMMER and the baseline CNN-LSTM model, under an embodiment.

FIGS. 13A-13H show Clarke Error Grid data, under an embodiment.

### DETAILED DESCRIPTION

### Example 1

Each of the following systems has distinct features and functionalities that contribute to more effective diabetes management, addressing different aspects of glucose monitoring and insulin delivery [1]-[3].
1) Self-Monitoring of Blood Glucose: Self-monitoring of Blood Glucose (SMBG) is essential in managing TID, allowing patients to regularly measure their blood glucose levels. This enables informed adjustments to diet, insulin therapy, and exercise, helping maintain optimal glucose control and prevent dysglycemia [5], [6]. However, SMBG provides only intermittent data, potentially missing significant glucose fluctuations. Additionally, excessive testing can lead to insulin stacking, increasing the risk of iatrogenic hypoglycemia. Proper patient education and adherence to monitoring schedules are crucial to maximize SMBG benefits while minimizing risks [7], [8].
2) Continuous Glucose Monitoring: Continuous Glucose Monitoring (CGM) systems offer real-time, continuous data on glucose levels by measuring concentrations in the interstitial fluid, closely aligning with plasma glucose values [9]. These systems include a sensor, transmitter, and receiver, providing frequent measurements that enhance glycemic control and enable timely interventions. Most FDA-approved CGM devices allow for non-adjunctive use, enabling therapeutic decisions without additional SMBG verification [10]. Despite their advantages, CGMs have a physiological delay of about 5-6 minutes between blood and interstitial glucose, necessitating occasional SMBG confirmation during rapid glucose changes [11]. In addition, patients must take this delay into account when making decisions about dosing insulin.
3) Hybrid Closed-loop Systems: Hybrid Closed-loop (HCL) systems integrate CGM data with automated insulin delivery, using algorithms to adjust insulin dosing in real-time based on continuous glucose readings [12]. This integration reduces the manual burden on patients and helps maintain glucose levels within the target range more effectively. Clinical trials have shown that HCL systems improve glycemic outcomes, such as lowering HbA1c levels and increasing time in range, while enhancing patients' quality of life by minimizing the need for constant monitoring and insulin adjustments [13]. As technology advances, HCL systems are expected to offer greater personalization and move closer to fully automated diabetes management solutions.
4) Automated Insulin Delivery Systems: Automated Insulin Delivery (AID) systems combine continuous subcutaneous insulin infusion (CSII) with CGM to dynamically regulate insulin delivery based on real-time glucose data [14]-[16]. Utilizing advanced algorithms, AID systems adjust basal insulin rates and administer corrective boluses to maintain glucose levels within desired ranges, reducing the risk of dysglycemia. Systems like the Tandem t: slim X2 with Control-IQ technology1 automate insulin delivery based on sensor data, significantly increasing time in range and decreasing extreme glucose events [17], [18]. Meta-analyses have demonstrated that AID systems improve glucose time in range (TIR) by substantial margins compared to conventional therapy, offering a more user-friendly and effective solution for daily diabetes management [19].

Despite advancements in diabetes management technologies, several limitations persist. AID systems often struggle to predict and react quickly to post-meal glucose spikes, delivering corrective boluses that are too slow or insufficient [20], [21]. While CGM systems provide real-time glucose data, they lack predictive features to warn users of impending 1https://www.tandemdiabetes.com/dysglycemia [22]. Both AID and HCL systems, although integrating CGM and insulin pumps, still require manual inputs for calibration and meals, limiting automation [13], [23]. SMBG, with intermittent testing, can miss critical glucose fluctuations, increasing the risk of undetected dysglycemia [6]. Additionally, the limited availability of clinical data and the high cost of collecting historical data for training create a cold-start issue for researchers.

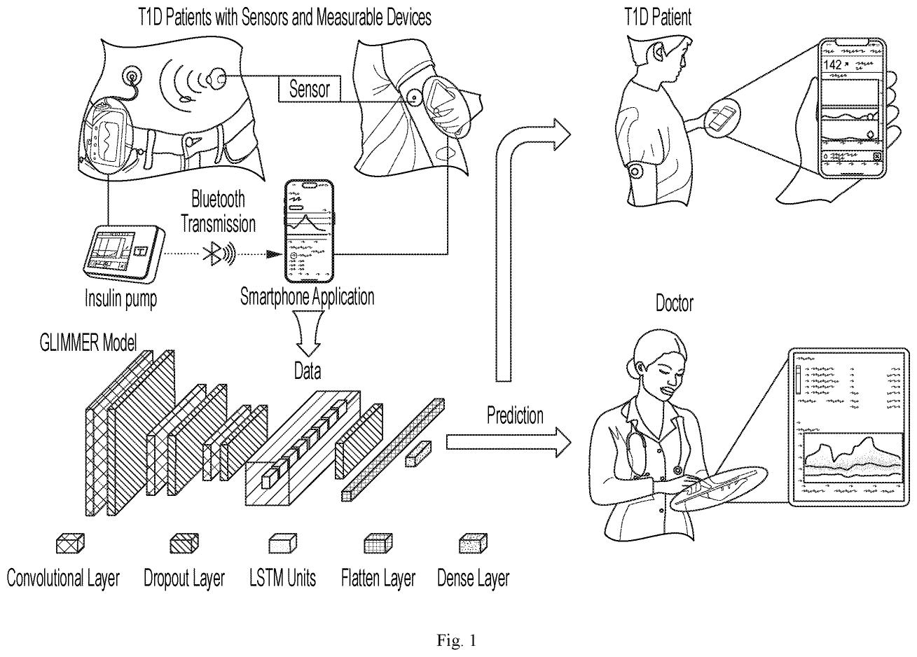

To address these limitations, we propose GLIMMER (Glucose Level Indicator Model with Modified Error Rate), an innovative machine learning model for continuous blood glucose forecasting. GLIMMER uses CGM data, insulin dosages, and meal inputs as key features and significantly improves prediction accuracy through a custom loss function that applies higher penalties in dysglycemic regions, effectively reducing errors in these critical areas and lowering overall prediction penalty to forecasting outcomes that are closely representing abnormal glucose events. By predicting glucose trends in advance, GLIMMER enhances AID systems, enabling proactive self-management behaviors and insulin adjustments. This allows AID systems to modify insulin delivery before glucose levels reach dangerous thresholds, mitigating physiological delays in glucose sensing and insulin action. Additionally, it allows patients to react to the prediction outcomes and actively engage in appropriate dietary, exercise, and insulin injection behaviors to prevent abnormal blood glucose events. As a result, GLIMMER creates opportunities to improve glycemic control and reduce the risk of adverse events, enhancing the safety and effectiveness of AID systems. In addition to developing an innovative glucose forecasting method, we collected approximately 26,707 hours of data from 25 patients with TID using AID systems. This newly created dataset enables the evaluation of our model in real-world scenarios and addresses the prevalent challenge of limited data availability for experimentation. An example of GLIMMER's practical application and its potential implementation in clinical practice is illustrated in FIG. 1.

FIG. 1 shows GLIMMER's application in a TID management system integrated with sensor technology, where data is sent directly to a smartphone or transmitted from a CGM device via Bluetooth or other methods. The data is then processed and used as input to the GLIMMER model, which predicts the next-hour of glucose levels and accurately reports potential dysglycemic events. This helps patients manage their condition more effectively and supports physicians in making informed decisions about the patient's treatment.

Advancements in forecasting future glucose levels have been crucial for managing patients with T1D, enabling them to proactively respond to glucose fluctuations and significantly improving glucose control. Marigliano et al. [24] demonstrated that integrating predictive alarms with CGM technology reduced hypoglycemic events in adolescents by 40% and severe hypoglycemia by 60%, highlighting the tangible benefits of predictive alerts in real-world settings. Vettoretti et al. [25] further supported these findings by showcasing how artificial intelligence-based diabetes management systems can enhance patient outcomes through early glucose predictions, prompting timely interventions such as insulin dose adjustments or dietary changes to maintain stable glucose levels and mitigate risks like cardiovascular disease and nerve damage. Additionally, Shroff et al. [26] emphasized the shift from reactive to proactive diabetes care with personalized prediction systems that learn individual response patterns, offering tailored alerts to meet each patient's unique physiological needs. These predictive technologies not only set a new standard in diabetes care by focusing on prevention over treatment but also significantly reduce the daily management burden. Arefeen et al. [27] suggest that machine learning algorithms can effectively predict hyperglycemia events using data from controlled feeding trials. In the following sections, we categorize these algorithms based on their architecture and discuss their approaches to predicting abnormal glucose levels, providing early warnings for timely interventions.

### Evidential Deep Learning and Meta-Learning

Machine learning techniques, particularly deep learning algorithms, have achieved reliable glucose-level prediction performance with minimal feature engineering required. Zhu et al. [28] demonstrated the use of evidential deep learning combined with meta-learning to create a model that adapts to individual patient data. This approach significantly enhances prediction accuracy by considering the uncertainty in predictions and personalizing the model to each patient's unique glucose response patterns. This method's strength lies in its ability to provide precise predictions with fewer input features, simplifying the data collection process for patients.

### Convolutional Recurrent Neural Networks

Another innovative approach is the use of Convolutional Recurrent Neural Networks (CRNN) [29] to estimate glucose levels for up to a 60-minute prediction horizon (PH) based on prior CGM data and information on meal and insulin intakes. Li et al. [30] introduced this model, which combines the feature extraction capabilities of convolutional neural networks (CNN) [31] with the temporal learning capabilities of recurrent neural networks (RNN) [32]. The CRNN model demonstrated superior performance in both simulated and real patient data, providing accurate short-term glucose predictions that are essential for proactive diabetes management.

### Long Short-Term Memory Networks

Long Short-Term Memory (LSTM) [33] units have also been employed by Aliberti et al. [34] to predict glucose levels. In this study, they developed a predictive model for blood glucose levels using a multi-patient dataset, focusing on leveraging the strengths of LSTM networks. The researchers compared the performance of LSTM networks with other models like Non-Linear Autoregressive (NAR) neural networks [35] and found that the LSTM model significantly outperformed others in both short- and long-term predictions. The LSTM model demonstrated superior accuracy due to its ability to handle long-term dependencies and mitigate issues like the vanishing gradient problem that commonly affects traditional RNNs. This study's findings underscore the potential of LSTM networks in enhancing predictive accuracy and clinical outcomes for diabetes management.

### Bi-Directional LSTM Variants

Further extending the capabilities of LSTM networks, researchers have explored bi-directional LSTM variants for glucose prediction [36]. Butt et al. [37] investigated how feature transformation techniques could enhance the efficiency of blood glucose prediction models. By employing bi-directional LSTM units, the model can consider both past and future data points, providing a more comprehensive understanding of glucose trends and improving prediction accuracy.

### Linear Regression

The Tandem t:slim X2 with Control-IQ technology employs simple linear regression to forecast blood glucose levels 30 minutes ahead based on previous CGM data, highlighting the importance of understanding these algorithms and their accuracy. To address the challenges of applying linear regression to time-series data and multi-step predictions, Zhang et al. [38]developed a multiple linear regression (MLR) model that predicts each future time step separately. This MLR approach combines k individual linear regression models, denoted as Lᵢ, each trained to relate the training data Xₜᵣₐᵢₙ to the CGM values at future time points yₜᵣₐᵢₙ(t+1) for i=1 to k. For instance, setting k=6 or 12 corresponds to predicting 30 or 60 minutes into the future, respectively. During the prediction phase, the trained models utilize their respective coefficients and intercepts to forecast glucose levels for the next k time steps by applying the models to the testing data one time step prior to the first test point. This iterative process is repeated for each row of the testing matrix, enabling the generation of multi-horizon predictions. Despite the inherent difficulties of using linear regression for timeseries forecasting, the structured MLR approach enhances the accuracy of glucose level predictions at various future points, thereby improving the reliability of automated insulin delivery systems. Additionally, this method allows for scalability and adaptability in different clinical settings, making it a valuable tool in the ongoing efforts to optimize diabetes management.

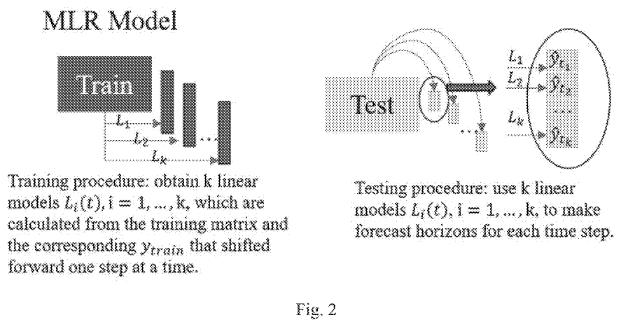

FIG. 2 shows a block diagram of the MLR model using multi Prediction Horizons (PH) [38].

### Methodology

### A. Model Architecture

While many machine learning algorithms are used for event forecasting, CNN-LSTM models excel in continuous multi-modal data by integrating spatially distributed data and capturing time-series patterns. These models are commonly applied in areas such as stock price forecasting, household load prediction, and wind power estimation [39]-[41]. Recent studies, including Jaloli and Cescon (2023) [42], have demonstrated that a CNN-LSTM model achieved a lower RMSE for glucose level predictions over a longer forecast horizon compared to other methods.

The decision to use a CNN-LSTM model for GLIMMER is rooted in its hybrid architecture, which combines the strengths of CNNs and LSTMs [31], [33]. The CNN component excels at automatic feature extraction, capturing spatial relationships within the data, while the LSTM component is adept at learning temporal sequences and long-term dependencies. This combination allows the CNN-LSTM model to effectively extract hidden features and correlations among various physiological variables, making it well-suited for forecasting future blood glucose values.

In comparative analyses, the CNN-LSTM model demonstrated superior performance over LSTM, CRNN, and other models, both in terms of predictive accuracy and clinical acceptability [42]. This improved performance is attributed to the model's sophisticated architecture, which includes stacks of convolutional and LSTM layers capable of learning complex, hidden features in multivariate datasets. The model's ability to capture rapid and abrupt changes in continuous glucose monitoring trends, due to its capacity to learn intricate dynamics and correlations between variables, further underscores its suitability for this task. However, it is important to note that the effectiveness of the CNN-LSTM model is contingent on the availability of a sufficiently large dataset, which also increases the computational cost compared to simpler reference models. To address the common issue of overfitting in LSTM networks, we incorporate dropout layers after each convolutional or LSTM layer, which has proven effective in enhancing model robustness [43].

### B. Custom Loss Function

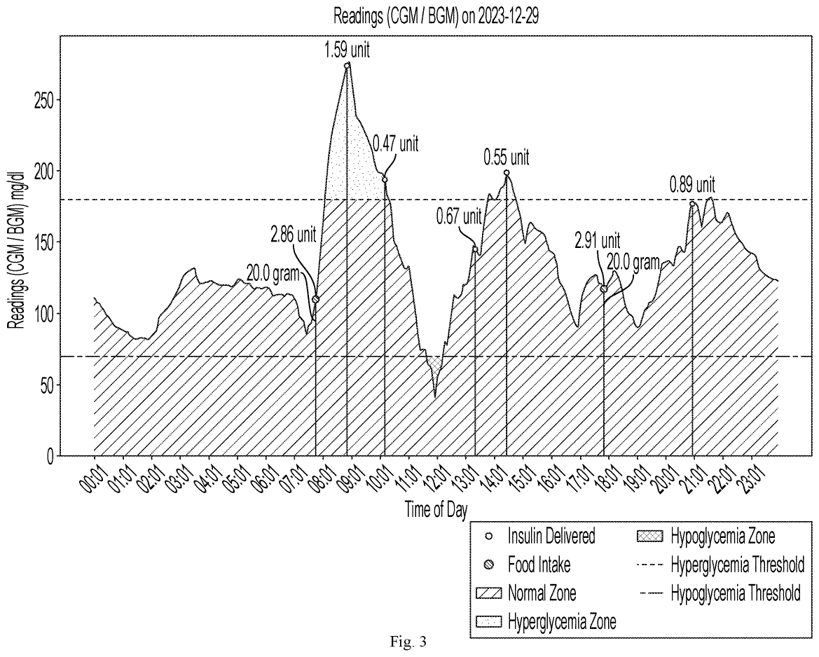

Glucose values can be categorized into normal and critical regions, with the latter including hyperglycemia and hypoglycemia thresholds. For patients with T1D, incorrect predictions in these critical regions can be particularly dangerous, resulting in missed intervention opportunities and potentially severe health complications. Despite extensive prior research on predicting glucose levels, a recent review by Woldaregay et al. [44] highlights a significant gap in the field. There is a noticeable lack of comprehensive analysis and modeling concerning the penalties for prediction errors across various dysglycemic regions. Therefore, we propose to penalize the prediction model for errors that occur in critical regions more heavily than those in normal regions. Assuming two thresholds T_{hypo} and T_{hyper} representing hypoglycemic threshold and hyperglycemia thresholds, respectively, we classify the blood glucose data x into three regions: 1 (hypoglycemia) for values below T_{hypo}, 2 (normal) for values between T_{hypo} and T_{hyper}, and 3 (hyperglycemia) for values above T_{hyper}. Commonly, insulin delivery devices and research articles set these thresholds to 70 mg/dL for hypoglycemia and 180 mg/dL for hyperglycemia [17]. Although these values may vary slightly from person to person, we use the general values commonly cited in similar studies [24], [25], leaving the indepth analysis to determine optimal thresholds for each patient for future research. FIG. 3 is an example of CGM readings data that highlights the regions and thresholds.

$$Glucose Level Regions = \{ \begin{matrix}1 , & x < {T}_{hypo} \\ 2 , & {T}_{hypo} \leq x \leq {T}_{hyper} \\ 3 , & x > {T}_{hyper}\end{matrix} \qquad ( 1 )$$

FIG. 3 shows CGM readings from a patient in the AZT1D dataset on Dec. 19, 2023, showing blood glucose fluctuations over 24 hours. Regions are labeled as follows: (1) Hypoglycemia (below 70 mg/dL, blue), (2) Normal range, and (3) Hyperglycemia (above 180 mg/dL, red). Dashed lines indicate hypoglycemia (blue) and hyperglycemia (red) thresholds.

We then break down the total error, which represents the cumulative prediction error across all data points, into individual errors within each critical region. By applying specific weights to these errors, our approach ensures that the model is more sensitive to inaccuracies where they matter most, enhancing its reliability and effectiveness in managing T1D. This total error, which we compute as the summation of individual losses, can be formalized as shown in (2):

$${Error}_{total} = {\sum }_{t = 1}^{3} {ω}_{i} \times {Error}_{i} \qquad ( 2 )$$

The total error serves as the model's loss function aggregated over all instances, enabling it to capture and prioritize prediction accuracy within distinct regions. If we choose Mean Absolute Error (MAE) as the error term Errorᵢ, and assign weights w_{hypo}, wₙₒᵣₘₐₗ, and w_{hyper} to the hypoglycemia, normal, and hyperglycemia regions, respectively, the total error can be calculated as follows:

$$M A E = \frac{1}{N} {\sum }_{i = 1}^{N} \vert {y}_{i} - {\hat{y}}_{i} \vert \qquad ( 3 )$$

$${Error}_{total} = \frac{{w}_{h y p o}}{{n}_{1}} {\sum }_{i = 1}^{{n}_{1}} \vert {y}_{i} - {\hat{y}}_{i} \vert + \frac{{w}_{n o r m a l}}{{n}_{2}} {\sum }_{i = 1}^{{n}_{2}} \vert {y}_{i} - {\hat{y}}_{i} \vert + \frac{{w}_{h y p e r}}{{n}_{3}} {\sum }_{i = 1}^{{n}_{3}} \vert {y}_{i} - {\hat{y}}_{i} \vert \qquad ( 4 )$$

where n₁, n₂ and n₃ represent the total number of blood glucose samples in each region, and yᵢ and ŷᵢ represent the predicted value and true value of the glucose level, respectively. The main research question that remains to be answered is how to determine the parameters of this error equation, including weight and threshold values.

### C. Error Weights

We classify the CGM values according to the threshold values discussed previously to identify the respective regions. Our next step is to finalize the custom loss function by determining the optimal weight parameters for each region, with a primary focus on dysglycemia areas. For this reason and to simplify the optimization problem, we set wₙₒᵣₘₐₗ to 1, reducing our optimization task to finding the optimal values for w_{hypo} and W_{hyper}To obtain optimal values of w_{hypo} and w_{hyper}, we propose to use Genetic Algorithms (GA) [45]. We chose this technique for several reasons. First, GAs are effective for optimization problems where the fitness function involves running another algorithm, particularly for complex or poorly defined problems. This is exactly the case in the problem at hand, finding the optimal weight values of w_{hypo} and W_{hyper} using the fitness function with the overall prediction error as output while also running a machine learning algorithm. Second, GAs do not need derivatives or extra information during the optimization process; they obtain the fitness score directly from the objective function [45]-[47]. Finally, it is straightforward to implement GAs, especially since we are also working on predicting CGM values with a CNN-LSTM network. Based on these considerations, we define our genetic algorithm's terminologies as follows:
Genome or Gene: A real number between [1, 10] representing a weight.
Chromosome or Individual Solution: A pair of weights (w_{hypo}, w_{hyper}).
Population: A pool of chromosomes of the size of N, each representing a candidate solution.
Crossover: In each generation, crossover is performed by averaging the values of two randomly selected parents to create a new child chromosome.
Mutation: The child chromosome undergoes mutation, where a small random perturbation is added to its values. This ensures diversity in the population.
Selection: The best individuals from the current population are combined with the new offspring to form the next generation, preserving strong solutions while introducing variations.
Fitness Function: The fitness function evaluates how well each chromosome performs by calculating the total error using the custom loss function. The fitness score is simply the value of this total error; lower fitness scores indicate better solutions. In each generation, the best scores are recorded, and the individuals with the lowest fitness scores are selected as the best individuals.

Algorithm 1 Finding the Best Pair of Weights for Each Patient

```
  1: Parameters:
  2: for each patient do
  3: Initialize population P with random weights w ∈ [1, 10]
  4: for g ← 1 to G do
  5: for each individual i ∈ P do
  6: evaluate fitness fᵢ
  7: end for
  8: sort population P by fitness
  9: P_{best} ← top N/2 individuals from P
10: P_{offspring} ← [ ]
11: while |P_{offspring}| < N/2 do
12: select parents p₁, p₂ ∈ P_{best}
13:  $c ← \frac{1}{2} ( {p}_{1} + {p}_{2} )$
14: m ← normal mutation ~ N (0, 0.5)
15: c ← clip(c + m, 1, 10)
16: P_{offspring} ← P_{offspring} ∪ c
17: end while
18: P ← P_{best} ∪ P_{offspring}
19: end for
20: w* ← arg min(fᵢ)
21: save w*
22: end for
```

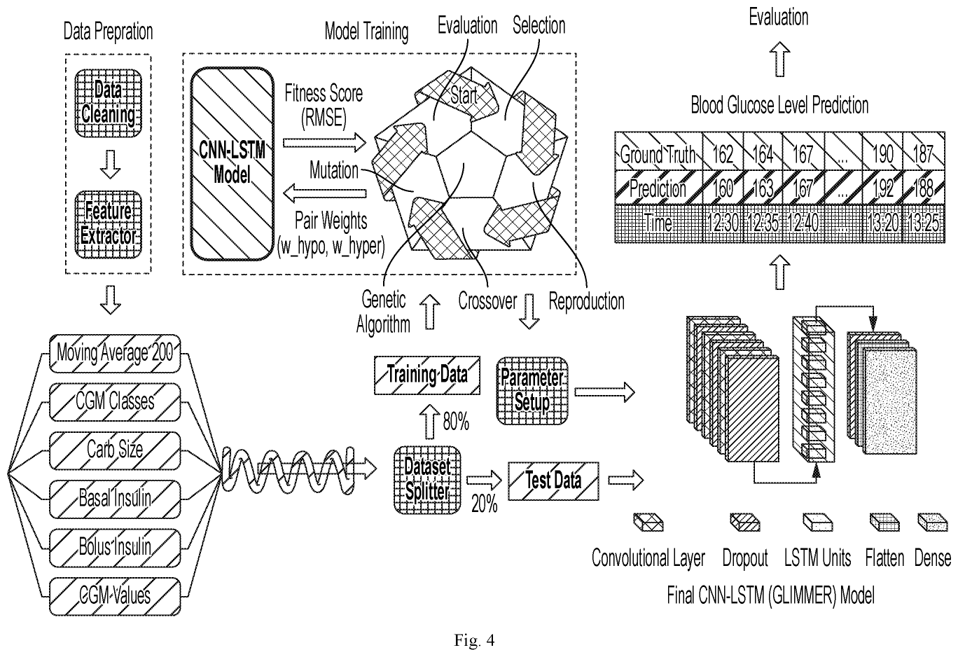

By iterating through multiple generations, the genetic algorithm refines the weight parameters, aiming to find the pair (w_{hypo}, w_{hyper}) that minimizes the error in critical regions. This process balances the need for generalization across all patients to optimize performance in dysglycemic regions. Algorithm 1 provides a high-level pseudo-code overview of the genetic algorithm process proposed to identify the best pair of weights. FIG. 4 illustrates the complete methodology for the design and evaluation of GLIMMER.

FIG. 4 shows the proposed methodology in GLIMMER for predicting blood glucose levels in patients with T1D. CGM data undergo preprocessing and feature extraction before being split for training and testing. A genetic algorithm optimizes parameters for a custom loss function, which is then used to train a CNN-LSTM model. Once trained, the custom loss function's parameters are finalized and applied to evaluate the model's performance using the test data.

### Evaluation Approach

### A. Dataset

1) OhioT1DM Dataset: We utilized the OhioT1DM dataset [48], which includes data from 12 individuals with T1D. The dataset contains raw glucose values recorded every 5 minutes, along with basal insulin, bolus insulin, and carbohydrate intake over an 8-week period.
2) AZT1D Dataset: In addition to using the OhioT1DM dataset, we also gathered data from 25 patients with T1D on AID systems who visited the endocrinology clinic at the Mayo Clinic in Scottsdale, AZ, between December 2023 and April 2024 for their regular appointments. Informed consents were taken from the recruited participants under the study named Machine Learning Design to Predict and Manage Postprandial Hyperglycemia in Patients with Type 1 Diabetes (IRB #23-003065). For each patient, the data includes, on average, 26 days of recordings collected in real-world settings, featuring CGM signals captured using Dexcom G6 Pro, insulin logs, meal carbohydrate sizes, and device modes (regular/sleep/exercise) obtained using Tandem t:slim X2 insulin pump. This dataset offers comprehensive insights into diabetes management by documenting various bolus events. These include standard boluses for meal-related carbohydrate intake, correction boluses for glucose adjustments, and automatic corrections made by the pump's algorithm. Each event is meticulously recorded, detailing insulin doses, target blood glucose levels, and user adjustments. This group of participants consists of 13 females and 12 males, aged between 27 and 80 years, with an average age of 59 years. This dataset contained 320,488 CGM entries, covering 26,707 hours of monitoring data.

### B. Data Preparation

The OhioT1DM dataset consists of 24 files containing data from 12 patients for training, validation, and testing. For each individual, there exist two files: one for training and one for testing [48]. To prepare the data for use in our evaluation, we chronologically partition the training file, using the first 80% for training and the remaining 20% for validation. This setup ensures that the validation dataset effectively evaluates the model after hyperparameter tuning without any risk of data leakage. The entire testing file is reserved for testing purposes. We apply a similar approach to the AZT1D dataset, first splitting the data of each patient into 80% for training and 20% for testing and then dividing the training set into 80% for training and 20% for validation. This method of partitioning has been widely utilized in previous studies on blood glucose prediction [28], [49]. In addition to the CGM values, basal insulin, bolus insulin, and carbohydrate amounts are included in the datasets.

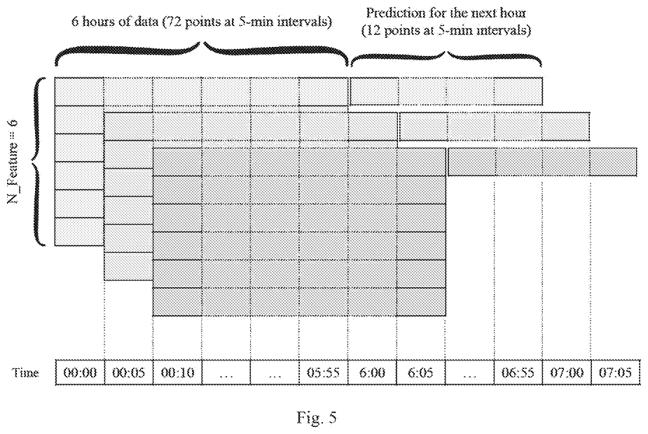

FIG. 5 shows that for time-series data, we treat it as sequential and use a sliding window to create samples for training a CNN-LSTM model. Since CGM values are recorded every five minutes, the window moves in five-minute steps, capturing both X (input) with 72 units and y (output) with 12 units. Each data unit includes 6 features. The figure above illustrates 3 data samples, each covering a 7-hour period.

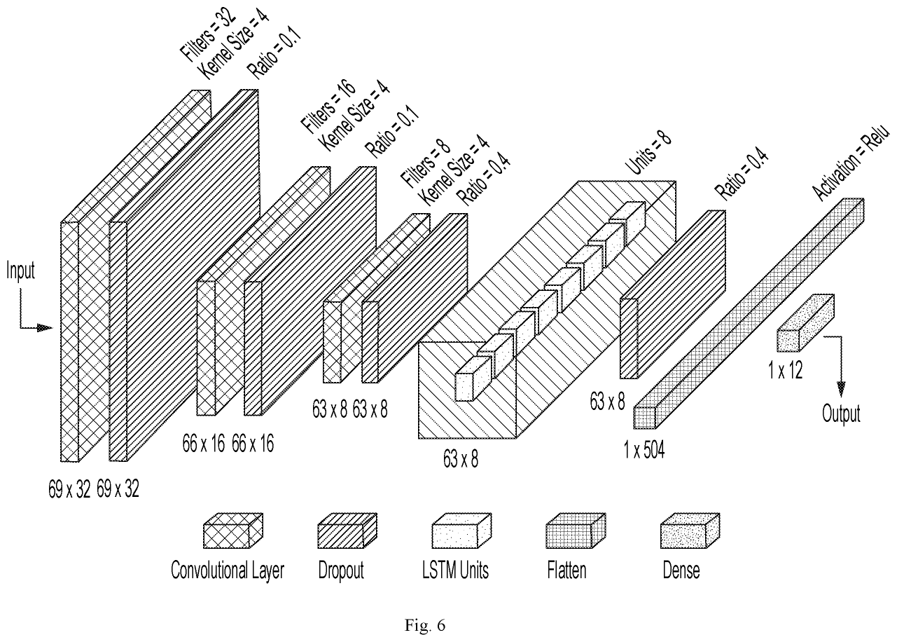

FIG. 6 shows a diagram of the proposed CNN-LSTM architecture. The input consists of 6 hours of time-series data with 6 features each, while the output provides a 1-hour prediction of glucose levels.

In addition to existing data that are used as features, we also added other features that might improve the model performance. Using the 200-period moving average as a feature in forecasting CGM values can be highly beneficial. In the context of CGM data, where glucose levels can fluctuate due to various factors like meals, physical activity, and stress, the 200-period moving average helps smooth out these short-term fluctuations. This smoothing effect allows the model to better capture the underlying long-term trends in glucose levels, which are crucial for making accurate predictions [50], [51]. By focusing on the long-term trend, the model is less likely to overfit short-term spikes or drops, thereby improving its generalization to new data. Another feature we implemented involves assigning a class label to CGM values. As discussed in Section III, we can categorize CGM values based on the hypoglycemia and hyperglycemia thresholds. We assign integer values (1, 2, or 3) to represent different regions: hypoglycemia, normal, and hyperglycemia. These region identifiers serve as an input feature to the model. Overall, the data preprocessing involved gathering 6 input features and applying a sliding window to the multivariate sequences, as shown in FIG. 5.

### C. Architecture Configuration

We reviewed previous studies to determine common configurations for CNN-LSTM models, focusing on the number of layers and LSTM units. From the insights gained in prior studies [52], [53], we identified a range of configurations, noting that these references utilized 1 to 4 convolutional layers and LSTM units in the range of 8 to 128. This understanding guided us in designing our experiment to systematically investigate the number of layers and units for our CNN-LSTM model. We utilized a subset of the dataset, dividing it into 80% for training and 20% for validation. We tested various configurations, including 1, 2, or 3 convolutional layers with 32, 16, and 8 filters and kernel sizes of 4 and 1 or 2 LSTM layers with 4, 8, 16, and 32 units. After each convolutional or LSTM layer, we applied a dropout layer with a rate of 0.1. The results of these experiments are shown in Table I. The optimal model configuration includes three 1D convolutional layers with kernel sizes of 4 and filter counts of 32, 16, and 8, respectively, to extract features from the input data. These features are then passed to an LSTM layer with 8 units for sequential processing. A flattening layer follows, preparing the features for a dense layer with a ReLU activation function that generates the final output. The proposed architecture is illustrated in FIG. 6.

### D. Custom Loss Function Parameters Configuration

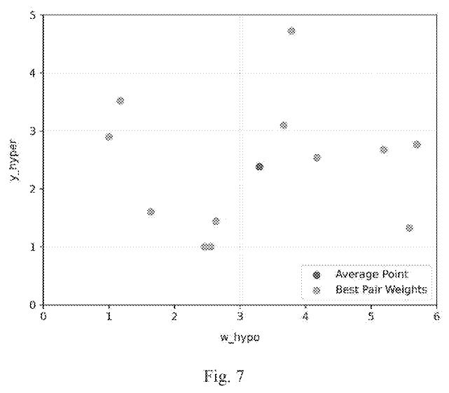

We designed an experiment and ran Algorithm 1 to find the best weights for our custom loss function. For each patient, we created a random population of 20 candidates, each with weight pairs in the range [1, 10], and evolved them over 25 generations following the methodology described in the Error Weights section. To simplify the process, we set wₙₒᵣₘₐₗ to 1 and focused on optimizing the two parameters, w_{hypo} and w_{hyper}. In each generation, we evaluated the performance of each candidate by setting the weights for the custom loss function and compiling and running our CNN-LSTM model. We then calculated the RMSE on the validation dataset, which served as the fitness score for each candidate. This process involved selecting the top-performing candidates to form the basis for the next generation, applying crossover and mutation operations to generate new candidates, and repeating the evaluation process. Ultimately, we identified the best weights for each patient. However, calculating the optimal weights for each individual patient is not feasible in a practical setting due to the time-consuming nature of running the genetic algorithm for each case. Instead, to provide a practical solution, we computed the average weights across all patients, resulting in (w_{hypo}, w_{hyper})=(3.296, 2.382). This average provides a balanced approach that generalizes well across patients while avoiding the inefficiencies of individual optimization. FIG. 7 shows the optimal weights for each patient alongside the average pair of weights. This method ensures a reasonable approximation of the custom loss function's performance without the impracticality of personalized optimization for each patient.

TABLE I
RMSE for CNN-LSTM Models with Various Configurations of Patient 559 from OhioT1DM Dataset

| | 1 LSTM Layer | | | | 2 LSTM Layers | | | |
|---|---|---|---|---|---|---|---|---|
| Convolutional Layers | 4 Units | 8 Units | 16 Units | 32 Units | 4 Units | 8 Units | 16 Units | 32 Units |
| 1 Layer (32 filters) | 38.62 | 31.48 | 34.22 | 33.94 | 33.04 | 31.90 | 43.78 | 43.02 |
| 2 Layers (32 and 16 filters) | 29.28 | 30.12 | 35.12 | 33.72 | 35.17 | 32.28 | 34.63 | 39.84 |
| 3 Layers (32, 16 and 8 filters) | 28.86 | 28.54 | 29.01 | 28.84 | 28.85 | 30.57 | 30.6 | 38.39 |

FIG. 7 shows the best pair of weights for all patients in OhioT1DM dataset and the average point.

### E. Experimental Setup

After data preparation and finalizing the GLIMMER model by setting up the parameters of the architecture and custom loss function, we conducted extensive experiments using a batch size of 48, over 30 epochs, with a prediction horizon (PH) of 60 minutes. To minimize the impact of randomness, we conducted 10 iterations of the experiments for each patient using a unique seed number for each run and reported the average results of these trials. The code used for these experiments is available for other researchers to reproduce our findings. All experiments were performed on an Apple M3 Pro chip featuring a 12-core CPU, an 18-core GPU, a 16-core Neural Engine, and 18 GB of unified memory.

### F. Evaluation Metrics

To assess the performance of the GLIMMER model, we employed standard metrics commonly used in related studies [28], [30], [34], [37], including:
1) Root Mean Square Error (RMSE): This metric provides insight into the average deviation of predicted values from actual values, with an emphasis on larger errors:

$$RMSE = \sqrt{\frac{1}{n} {\sum }_{i = 1}^{n} {( {y}_{i} - {\hat{y}}_{i} )}^{2}} \qquad ( 5 )$$

Here, n represents the number of data points, yᵢ denotes the ground truth or actual CGM value, and ŷᵢ is the predicted CGM value.
2) Mean Absolute Error (MAE): This metric quantifies the average absolute difference between predicted and observed values, providing a straightforward interpretation of prediction accuracy. It is calculated as follows:

$$M A E = \frac{1}{n} {\sum }_{i = 1}^{n} \vert {y}_{i} - {\hat{y}}_{i} \vert \qquad ( 6 )$$

3) Precision: This metric indicates the accuracy of the positive predictions made by the model, defined as the ratio of true positives (TP) to the sum of true positives and false positives (FP). It is calculated as:

$$Precision = \frac{T P}{T P + F P} \qquad ( 7 )$$

4) Recall: This metric reflects the model's ability to identify all relevant instances, representing the true positive rate. It is the ratio of true positives to the sum of true positives and false negatives (FN):

$$Recall = \frac{T P}{T P + F N} \qquad ( 8 )$$

5) F1 Score: The F1 score provides a balance between precision and recall, especially useful when dealing with imbalanced classes. It is calculated as:

$$F 1 = 2 \cdot \frac{Precision \cdot Recall}{Precision + Recall} \qquad ( 9 )$$

6) Clarke Error Grid Analysis: The Clarke Error Grid (CEG) analysis [54] is a widely accepted tool for evaluating the clinical accuracy of glucose predictions by comparing them to reference glucose values. It classifies predictions into five distinct regions, each representing varying levels of clinical significance:
Region A: Includes values within 20% of the reference value, indicating clinically accurate predictions.
Region B: Contains values outside of the 20% range but unlikely to result in inappropriate treatment.
Region C: Identifies predictions that may lead to unnecessary treatment.
Region D: Represents predictions where critical hypoglycemia or hyperglycemia might be missed, posing a potential danger.
Region E: Captures predictions that could lead to confusion between treating hypoglycemia and hyperglycemia, a highly dangerous scenario.

### Results

Tables II and III summarize the performance comparison of GLIMMER with other methods for the OhioT1DM and AZT1D datasets, both using a PH of 60 minutes. We selected the most recent studies that employed a variety of classical and machine learning models, including Fast-Adaptive and Confident Neural Network (FCNN) [28], CRNN [30], Bi-LSTM [36], transformer models [55], Random Forest Regression (RFR) [28], [56], Support Vector Regression (SVR) [57], and Autoregressive Integrated Moving Average (ARIMA) [58]. Additionally, we included the GLIMMER model without modifications, represented as a basic CNN-LSTM, to highlight the effects of our enhancements, which incorporate two crafted features and a custom loss function.

The results include RMSE and MAE, presented as (Mean±Standard Deviation), along with CEG reports, which serve as standard error metrics for predictions. For both RMSE and MAE, lower values indicate better performance. As shown in Table II, GLIMMER demonstrates outstanding performance compared to other models, achieving a 23% improvement in RMSE and a 31% improvement in MAE relative to the best-reported errors. The basic CNN-LSTM results further indicate its potential as a viable candidate for analyses where other models may not be applicable. In the CEG analysis, higher values in Region A are preferable, while lower values are desirable in other regions. Notably, GLIMMER's predictions achieve 85% within Region A, yielding a 15% improvement compared to FCNN, and it also maintains one of the lowest values in other regions.

Table III presents the results for GLIMMER, and the basic CNN-LSTM model applied to the AZT1D dataset. GLIMMER again outperforms the basic CNN-LSTM model, achieving a 24% improvement in RMSE and a 28% improvement in MAE. Since this dataset has been recently collected and is not publicly available, we could only generate results for these two models, leaving evaluations for other methods to future researchers. However, given the close results in Table II for the basic CNN-LSTM and leading models like FCNN and CRNN, we anticipate similar outcomes.

To ensure a fair comparison, we did not include the MLR model in Table II, as this model employs a multi-model approach that requires training a separate model for each PH. In our case, with a PH of 60 minutes, this necessitates the creation of 12 individual models to predict the next 5, 10, 15, . . . , 55, and 60 minutes. In contrast, all the methods presented in Table II utilize a single model for their predictions, and the MLR model did not report CEG analysis in their studies. While they achieved an RMSE of 24.58 mg/dL and an MAE of 17.42, which are better than those of some other models, GLIMMER still outperformed them.

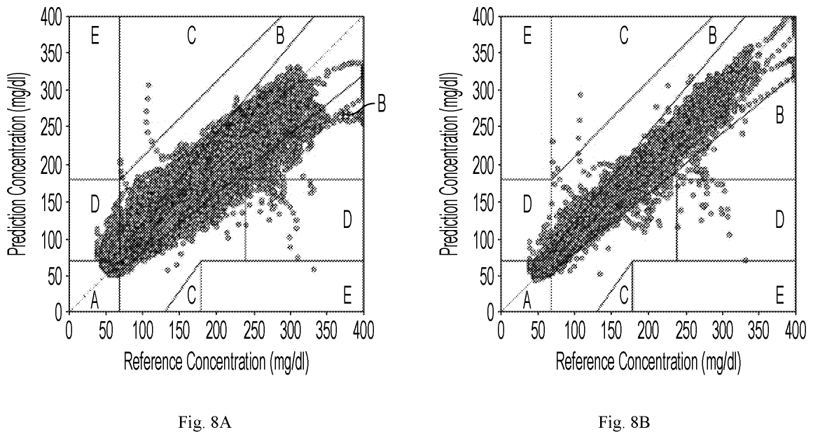

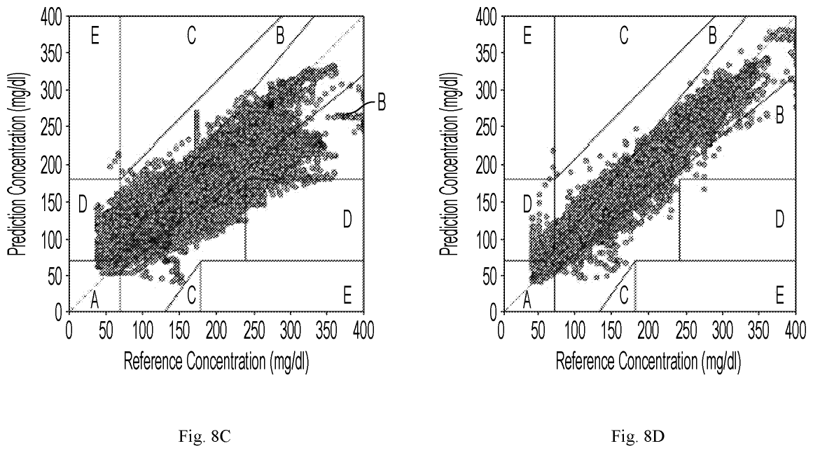

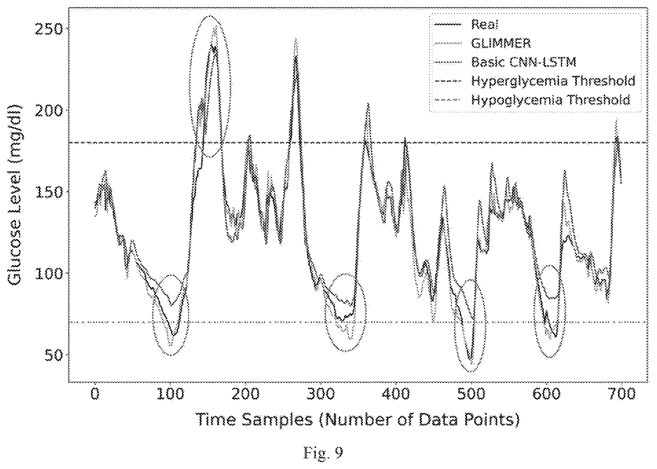

We visualized the results of the CEG analysis for both the OhioT1DM and AZT1D datasets in FIGS. 8A-8D. The enhanced prediction accuracy of the GLIMMER model compared to the basic CNN-LSTM is evident, demonstrating its ability to accurately forecast blood glucose levels even in critical regions, which is crucial for clinical analysis and real-world applications. Additionally, FIG. 9 illustrates the glucose level predictions of GLIMMER compared to the basic CNN-LSTM. This figure highlights GLIMMER's ability to accurately predict blood glucose levels, particularly during peaks, due to the integration of the custom loss function. The consistent results across the OhioT1DM and AZT1D datasets further demonstrate that GLIMMER is robust and generalizable, making it suitable for use with various datasets.

TABLE II
Prediction Performance Comparison on the
OhioT1DM Dataset with PH ± 60 minutes

| | RMSE | MAE | CEG-Regions (%) | | | | |
|---|---|---|---|---|---|---|---|
| Model Name | (mg/dL) | (mg/dL) | A | B | C | D | E |
| FCNN [28] | 31.07 ± | 22.86 ± | 72.58 ± | 24.39 ± | 0.16 ± | 2.85 ± | 0.02 ± |
| | 3.62 | 2.89 | 7.87 | 6.41 | 0.14 | 1.68 | 0.04 |
| CRNN [30] | 32.02 ± | 23.82 ± | 71.06 ± | 25.57 ± | 0.15 ± | 3.20 ± | 0.01 ± |
| | 3.76 | 3.13 | 8.69 | 7.07 | 0.17 | 1.99 | 0.04 |
| Bi-LSTM [36] | 33.44 ± | 24.59 ± | 70.61 ± | 25.98 ± | 0.17 ± | 3.19 ± | 0.05 ± |
| | 3.76 | 2.89 | 8.21 | 6.70 | 0.13 | 1.89 | 0.07 |
| Transformer [55] | 32.96 ± | 24.19 ± | 71.70 ± | 25.20 ± | 0.15 ± | 2.92 ± | 0.04 ± |
| | 3.70 | 2.79 | 7.77 | 6.43 | 0.15 | 1.65 | 0.05 |
| SVR [57] | 33.83 ± | 25.63 ± | 66.43 ± | 29.61 ± | 0.20 ± | 3.73 ± | 0.03 ± |
| | 3.62 | 2.98 | 9.15 | 7.30 | 0.21 | 2.62 | 0.04 |
| RFR [56] | 35.31 ± | 26.43 ± | 67.03 ± | 29.38 ± | 0.23 ± | 3.34 ± | 0.02 ± |
| | 3.72 | 3.02 | 8.17 | 6.29 | 0.19 | 2.14 | 0.04 |
| ARIMA [58] | 35.42 ± | 25.97 ± | 68.77 ± | 28.65 ± | 0.46 ± | 2.06 ± | 0.05 ± |
| | 3.74 | 2.70 | 6.85 | 5.83 | 0.40 | 1.00 | 0.05 |
| Basic CNN-LSTM | 31.98 ± | 23.00 ± | 74.31 ± | 23.12 ± | 0.11 ± | 2.43 ± | 0.03 ± |
| | 4.15 | 2.87 | 7.03 | 5.96 | 0.14 | 1.56 | 0.08 |
| GLIMMER | 23.97 ± | 15.83 ± | 85.46 ± | 13.26 ± | 0.13 ± | 1.12 ± | 0.02 ± |
| | 3.77 | 2.09 | 4.87 | 4.29 | 0.17 | 0.57 | 0.07 |

TABLE III
Prediction Performance Comparison on the AZT1D Dataset with PH = 60 minutes

| | RMSE | MAE | CEG-Regions (%) | | | | |
|---|---|---|---|---|---|---|---|
| Model Name | (mg/dL) | (mg/dL) | A | B | C | D | E |
| Basic CNN-LSTM | 29.55 ± | 21.61 ± | 73.27 ± | 24.74 ± | 0.03 ± | 2.21 ± | 0.02 ± |
| | 6.49 | 5.19 | 8.57 | 7.65 | 0.07 | 1.67 | 0.05 |
| GLIMMER | 22.48 ± | 15.58 ± | 83.89 ± | 14.94 ± | 0.02 ± | 1.12 ± | 0.02 ± |
| | 3.57 | 2.87 | 5.01 | 4.54 | 0.03 | 0.85 | 0.04 |

FIGS. 8A-8D shows the Clarke Error Grid for the basic CNN-LSTM model and GLIMMER across all patients. FIGS. 8A and 8B pertain to the OhioT1DM dataset (with FIG. 8A for the basic CNN-LSTM and Fig. B for GLIMMER), while FIGS. 8C and 8D relate to the AZT1D dataset (with FIG. 8C for the basic CNN-LSTM and FIG. 8D for GLIMMER). The detailed percentages for each region are represented in Tables II and III. In both datasets, the results are tightly clustered near the x=y line, indicating that GLIMMER's predictions are as close as possible to the reference values.

FIG. 9 shows a forecasting comparison between GLIMMER and the basic CNN-LSTM model. The solid black line represents CGM values from patient 552 in the OhioT1DM test dataset, covering 700 data points at 5-minute intervals. The dashed gray and black lines indicate hypoglycemia and hyperglycemia thresholds at 70 mg/dL and 180 mg/dL, respectively. The red circles highlight the prediction accuracy of the two models, especially during peak values in critical regions.

In our analysis, we calculated error metrics separately for normal glucose levels and dysglycemic regions, providing a clearer understanding of the model's performance across different glucose conditions. This detailed breakdown highlights the model's strengths and identifies areas for improvement in managing varying glucose states. Precision and recall offer valuable insights: high precision indicates that the model accurately predicts dysglycemia, reducing false alarms, while high recall shows that the model effectively detects dysglycemic events, minimizing missed occurrences.

Tables IV and V present these metrics for the OhioT1DM and AZT1D datasets, comparing the performance of the basic CNN-LSTM model and GLIMMER. These metrics, along with RMSE and MAE, provide a comprehensive assessment of the model's reliability and effectiveness in supporting diabetes management and patient safety. In the OhioT1DM dataset, GLIMMER significantly improves hypoglycemia detection, increasing recall from 16% to 42%, meaning it captures more true low blood sugar events, which is vital for patient safety. The F1 score for hypoglycemia rises from 35% to 44%, reflecting a better balance between detecting true events and minimizing false positives. For hyperglycemia, GLIMMER also improves recall by 10%, from 78% to 86%, ensuring more high blood sugar episodes are detected. With a 5% improvement in precision, it reduces false alarms, making the model more reliable and user-friendly in managing glucose levels. In the AZT1D dataset, GLIMMER shows notable gains in both recall and precision for hypoglycemia detection. Recall improves from 2% to 13%, meaning it catches more low blood sugar events, while precision rises from 34% to 47%, reducing unnecessary alerts. For hyperglycemia, GLIMMER's recall jumps from 48% to 73%, a 52% improvement, allowing it to detect more high glucose events. The model also enhances precision by 15%, ensuring better accuracy in its predictions, making it a more effective tool for managing critical dysglycemic events.

Our experiments demonstrated that GLIMMER achieves superior performance and accuracy as a predictive model, surpassing the state-of-the-art models. Additionally, CEG analysis validated its reliability in detecting and forecasting dysglycemic events. Recent work by Annuzzi et al. [59] explored how certain features affect blood glucose prediction using XAI methodologies. They reported an RMSE of 24.47±4.27 on the AI4PG dataset, with a PH of 60 minutes. Their model depends on detailed data inputs, including preprandial blood glucose levels, insulin dosages, and meal-related factors such as energy intake, macro-nutrients, glycemic index, and glycemic load.

However, we note that in real-world scenarios, obtaining these details can be challenging, as it often requires manual logging of meal and nutritional information. GLIMMER, on the other hand, achieves strong predictions using only features like CGM data, bolus and basal insulin levels, and carbohydrate amounts that are commonly obtained in automated insulin delivery systems by default. As a result, GLIMMER does not impose any additional data collection burden on the patients beyond what the standard of care requires.

TABLE IV
Performance comparison between GLIMMER and the basic CNN-LSTM model in
different glucose regions of the OhioT1DM dataset with PH = 60 minutes.

| | GLIMMER | | | | Basic CNN-LSTM | | | |
|---|---|---|---|---|---|---|---|---|
| Metrics | Normal | Dysglycemia | Hyperglycemia | Hypoglycemia | Normal | Dysglycemia | Hyperglycemia | Hypoglycemia |
| RMSE | 21.46 ± | 28.06 ± | 28.77 ± | 24.61 ± | 26.43 ± | 39.34 ± | 40.58 ± | 36.24 ± |
| (mg/dL) | 3.29 | 6.06 | 7.28 | 6.98 | 2.46 | 7.92 | 9.77 | 9.04 |
| MAE | 14.43 ± | 18.54 ± | 19.12 ± | 18.45 ± | 19.54 ± | 29.33 ± | 30.00 ± | 30.41 ± |
| (mg/dL) | 2.14 | 3.18 | 4.05 | 8.22 | 1.90 | 6.03 | 7.61 | 9.21 |
| F1 Score | 94.00 ± | 91.00 ± | 85.00 ± | 44.00 ± | 94.00 ± | 85.00 ± | 78.00 ± | 35.00 ± |
| (%) | 1.00 | 4.00 | 4.00 | 23.00 | 2.00 | 5.00 | 6.00 | 19.00 |
| Recall | 100.00 ± | 100.00 | 86.00 ± | 42.00 ± | 100.00 ± | 100.00± | 78.00 ± | 16.00 ± |
| (%) | 0.00 | 0.00 | 5.00 | 29.00 | 0.00 | 0.00 | 7.00 | 21.00 |
| Precision | 89.00 ± | 84.00 ± | 83.00 ± | 46.00 ± | 88.00± | 74.00 ± | 79.00 ± | 34.00 ± |
| (%) | 2.00 | 6.00 | 5.00 | 23.00 | 3.00 | 8.00 | 9.00 | 27.00 |

TABLE V
Performance comparison between GLIMMER and the basic CNN-LSTM model in
different glucose regions of the AZT1D dataset with PH = 60 minutes.

| | GLIMMER | | | | Basic CNN-LSTM | | | |
|---|---|---|---|---|---|---|---|---|
| Metrics | Normal | Dysglycemia | Hyperglycemia | Hypoglycemia | Normal | Dysglycemia | Hyperglycemia | Hypoglycemia |
| RMSE | 19.85 ± | 31.63 ± | 31.75 ± | 41.69 ± | 23.63 ± | 46.42 ± | 46.82 ± | 57.82 ± |
| (mg/dL) | 3.18 | 6.15 | 9.41 | 21.73 | 4.52 | 10.55 | 13.40 | 21.75 |
| MAE | 13.99 ± | 22.86 ± | 23.43 ± | 36.15 ± | 17.95 ± | 37.28 ± | 37.97 ± | 54.89 ± |
| (mg/dL) | 2.51 | 4.68 | 8.95 | 20.69 | 4.04 | 9.43 | 13.09 | 22.32 |
| F1 Score | 96.00 ± | 82.00 ± | 74.00 ± | 22.00 ± | 97.00 ± | 58.00 ± | 54.00 ± | 11.00 ± |
| (%) | 1.00 | 11.00 | 9.00 | 20.00 | 3.00 | 25.00 | 21.00 | 13.00 |
| Recall | 100.00 ± | 100.00 ± | 73.00 ± | 13.00 ± | 100.00 ± | 100.00± | 48.00 ± | 2.00 ± |
| (%) | 0.00 | 0.00 | 5.00 | 20.00 | 0.00 | 0.00 | 25.00 | 5.00 |
| Precision | 93.00 ± | 71.00 ± | 72.00 ± | 47.00 ± | 94.00 ± | 45.00 ± | 68.00 ± | 49.00 ± |
| (%) | 3.00 | 14.00 | 9.00 | 26.00 | 5.00 | 24.00 | 18.00 | 37.00 |

Despite these achievements, certain limitations remain. While GLIMMER shows improvement in F1 score, precision, and recall over the baseline CNN-LSTM model, its hypoglycemia prediction performance is still limited. One reason for this limitation is the sparsity of hypoglycemic events in both the OhioT1DM and AZT1D datasets used in this study. The scarcity of these events means the algorithm has fewer examples from which to learn hypoglycemia patterns effectively. Additionally, we were unable to compare GLIMMER to other models on the AZT1D dataset because the source codes for these methods are not publicly available. In future work, we plan to implement and evaluate additional models to achieve a more comprehensive assessment.

Although the core architecture of GLIMMER is CNN-LSTM, the methodologies presented in this article, including the proposed custom loss function and the optimization algorithm, are model-agnostic and can be adapted to other neural network models as well. Future work will focus on designing more advanced architectures, such as attention-based models, to explore GLIMMER's potential within more complex timeseries forecasting frameworks. We also aim to extend the prediction horizon and incorporate long-term features, which could improve accuracy over longer time periods. As shown in FIG. 1, integrating GLIMMER with automated insulin delivery devices and developing a smartphone application to alert patients and physicians about potential dysglycemic events could have transformative effects. This application would provide projections for the next hour of blood glucose levels, acting as a preventive tool and allowing for a comparison of GLIMMER's effectiveness against current methods.

The GLIMMER model described herein comprises a machine learning algorithm with a custom loss function designed for accurate prediction of blood glucose levels and to create more reliable opportunities for behavioral and medical treatments in type 1 diabetes management. Our contribution emphasizes predictions in dysglycemic regions, where patients face dangerous conditions and require precise forecasts to prevent adverse events. Utilizing carefully selected input features and a custom loss function fine-tuned through a genetic algorithm, GLIMMER has demonstrated improved performance over state-of-the-art models, reducing RMSE by 23% and MAE by 31% on the OhioT1DM dataset. Additionally, we collected a new dataset containing CGM records and insulin delivery events from 25 patients with T1D, allowing us to validate GLIMMER's generalizability on a larger, real-world dataset while also creating a valuable resource for further research. GLIMMER can be integrated into automated insulin delivery systems and smartphone applications, supporting patients and physicians in more accurately managing TID and preventing dysglycemia.

### Example 2

### 1 Introduction

Type 1 diabetes (T1D) is an autoimmune condition in which insulin-producing beta cells in the pancreas are destroyed, leading to lifelong dependence on exogenous insulin. An estimated 8.4 million people worldwide live with T1D, accounting for roughly 5-10% of all diabetes cases [1]. The prevalence of T1D varies significantly across populations and regions, highlighting the need for tailored management strategies to support diverse patient needs globally. Managing T1D is challenging due to the need for constant monitoring and precise insulin dosing to maintain blood glucose within a safe range. Poor glucose control can result in serious complications, including cardiovascular disease, neuropathy, retinopathy, and kidney failure [2-4].

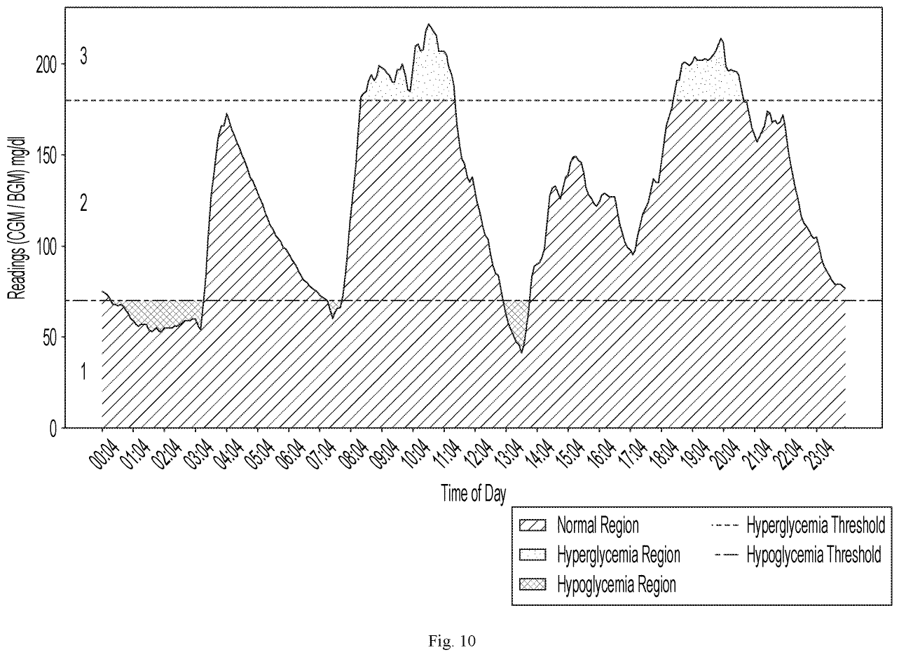

Predicting future blood glucose levels is essential for preventing dangerous dysglycemic events (hypoglycemia and hyperglycemia) and supporting timely, proactive interventions focused on insulin dosing and behavioral modifications (e.g., changes in diet, activity, sleep) [5]. It is well established that in AI-powered glucose forecasting models, errors occurring in critical regions-such as misclassifying an impending hyperglycemic event as normal-pose significant health risks [6-9]. In contrast, prediction deviations that remain within the target glucose range (70-180 mg/dL) are generally less consequential [10]. FIG. 10 depicts the clinically defined hypoglycemic, normal, and hyperglycemic regions.

FIG. 10 shows blood glucose readings captured every 5 minutes. Regions are labeled as follows: (1) Hypoglycemia (below 70 mg/dL, blue), (2) Normal range, and (3) Hyperglycemia (above 180 mg/dL, red). Dashed lines indicate hypoglycemia (blue) and hyperglycemia (red) thresholds.

To fully leverage predictive capabilities in real-world settings, these models can be integrated into modern diabetes management technologies. Continuous glucose monitoring (CGM) devices measure interstitial glucose concentrations in real-time, providing frequent data that closely track blood glucose trends and support timely clinical decisions [11]. Automated insulin delivery (AID) systems integrate CGM data with insulin pumps to dynamically regulate insulin dosing using [12]. Hybrid closed-loop (HCL) systems, a subset of AID technologies, automate basal insulin delivery based on real-time sensor input [13]. When predictive models are embedded within these technologies, they can help overcome the physiological delays of insulin action and glucose sensing, enabling earlier alerts or therapeutic adjustments before glucose levels enter dangerous ranges [14, 15].

Despite advancements in diabetes management technologies, several limitations persist. Existing glucose prediction models often rely on generic architectures and overlook the domain-specific complexities of T1D management. Many models fail to maintain accuracy in clinically significant dysglycemic regions, particularly during rapid fluctuations such as postprandial glucose spikes, where timely intervention is critical [16-20]. As summarized in Table VI, these limitations highlight the need for domain-specific, clinically-aware designs that prioritize safety-critical objectives. Another key challenge lies in data availability: the high cost and burden of collecting detailed, labeled data limit access to large, high-quality datasets [21]. This scarcity creates a cold-start problem for researchers and impedes both model generalizability and personalization [22].

To address these challenges, we propose GLIMMER (Glucose Level Indicator Model with Modified Error Rate), a domain-specific prediction framework designed for T1D management. Our approach combines tailored feature engineering, extensive hyperparameter tuning, and a novel custom loss function that prioritizes accuracy in clinically-critical blood glucose regions by penalizing errors that occur in hyperglycemic and hypoglycemic regions. We formulate the problem of finding optimal value of the penalty weight for each region (i.e., hyperglycemia, hypoglycemia, and normo-glycemia) as an optimization problem over the training loss. We then devise a genetic algorithm to learn optimal values of the weights, placing greater emphasis on errors during dysglycemic events. We show that this strategy is architecture-agnostic and can be effectively applied across commonly used forecasting models, including Long Short-Term Memory (LSTM) and Transformer-based networks. To support evaluation beyond existing datasets, we conduct a real-world study and introduce the AZT1D dataset, which comprises 26,707 hours of multimodal data from 25 individuals with T1D [26]. This new dataset enables robust evaluation on a distinct cohort and is used in this study to demonstrate the effectiveness of GLIMMER across two independent datasets.

TABLE VI
Summary of Related Models in Blood Glucose Forecasting

| Model | Key Features | Limitations |
|---|---|---|
| FCNN | Simple fully connected | Does not model temporal |
| [22] | network; fast to train and | dynamics; limited capacity to |
| | deploy. | capture glucose trends over |
| | | time. |
| CRNN | Combines CNN and RNN | General-purpose architecture; |
| [16] | layers to learn both spatial and | lacks focus on clinical events |
| | temporal features. | like hypoglycemia and |
| | | hyperglycemia. |
| LSTMs | Captures long-term | Prioritizes average error; lacks |
| [17, 18] | dependencies in time-series; | domain-specific loss functions |
| | bidirectional versions use | to highlight critical regions. |
| | future context. | |
| MLR | Multi-step extension of linear | Poor at modeling nonlinear |
| [23] | regression; interpretable and | interactions and long-range |
| | fast. | dynamics; limited scalability. |
| Informer | Efficient Transformer variant | Requires significant training |
| [19] | for long-range time-series | data; not tailored for glycemic |
| | prediction. | event sensitivity. |
| TimesNet | Learns temporal variations in | High model complexity; lacks |
| [20] | time-series via 2D | explicit clinical |
| | decomposition. | interpretability. |
| Gluformer | Transformer-based model | Strong accuracy but still data- |
| [24] | with uncertainty | hungry and resource- |
| | quantification. | intensive. |
| BG-BERT | Self-supervised Transformer | Large parameter count; |
| [25] | for contextual glucose | computationally demanding |
| | prediction. | for edge deployment. |

### 2 Related Work

Machine learning and deep learning approaches have significantly advanced blood glucose prediction, enabling proactive interventions and improved glycemic control for patients with T1D. Marigliano et al. [9] demonstrated that integrating predictive alarms with CGM technology reduced hypoglycemic events in adolescents by 40% and severe hypoglycemia by 60%, highlighting the clinical benefits of early warnings. Similarly, Vettoretti et al. [27] showed how AI-based systems can improve outcomes by prompting timely insulin adjustments or dietary changes. In line with these efforts, Arefeen et al. [28] developed predictive models for postprandial hyperglycemia using controlled feeding trial data.

To improve predictive performance, researchers have explored a range of architectures. Zhu et al. [22] applied fully connected neural networks (FCNNs) for their simplicity and speed, while El Khatib et al. [29] used convolutional models (CNNs) to extract local patterns in CGM trends. Li et al. [16] proposed a hybrid CRNN model to capture both spatial and temporal glucose dependencies. Aliberti et al. [17] and Lu et al. [30] demonstrated how LSTM-based models can capture long-term temporal patterns, and Sun et al. [18] used Bi-LSTM to incorporate future context for improved forecasting. Simpler models such as ARIMA [31] and MLPs [32] are still used for interpretability and computational efficiency.

Transformer-based architectures have recently gained attention due to their ability to model long-range dependencies and parallelize computation. Informer [19] and TimesNet [20] have shown strong performance in long-horizon glucose prediction. Gluformer [24] enhances this line of work by incorporating uncertainty quantification, while BG-BERT [25] applies self-supervised learning to extract contextual information from time-series data. Despite their effectiveness, such models often require large training datasets and substantial computational resources, making them less feasible for deployment on memory- and power-constrained edge devices.

### 3 Methodology

### 3.1 Model Architecture

The GLIMMER framework is architecture-agnostic, designed to enhance blood glucose forecasting regardless of the underlying model architecture. To demonstrate this flexibility, we applied GLIMMER to two representative architectures: CNN-LSTM and Transformer. These choices reflect both established and state-of-the-art modeling approaches commonly used in the domain of time-series glucose prediction.

The CNN-LSTM architecture combines the feature extraction strengths of convolutional layers with the temporal modeling capabilities of LSTM units. This hybrid design effectively captures spatial and sequential patterns within multivariate physiological data. Prior work by Jaloli and Cescon [33] showed that CNN-LSTM outperforms LSTM, CRNN, and other baselines in predictive accuracy and clinical relevance, especially over longer horizons. In parallel, Transformer-based models have shown strong potential for modeling long-range dependencies via self-attention and have become increasingly popular in time-series forecasting [19, 20, 24]. By evaluating GLIMMER with both architectures, we highlight its ability to generalize across modeling paradigms while improving performance in critical glycemic regions.

### 3.2 Custom Loss Function

Glucose values are typically divided into three clinically meaningful regions: hypoglycemia, normal, and hyperglycemia as shown in (1). Values below the hypoglycemia threshold T_{hypo} indicate dangerously low glucose levels, while values above the hyperglycemia threshold T_{hyper} indicate excessively high levels. Measurements that fall between these two thresholds are considered within the normal range. In practice, T_{hypo} and T_{hyper} are often set to 70 mg/dL and 180 mg/dL, respectively, based on guidelines and prior studies [9, 27, 34].

Prediction errors in the critical regions of hypo- and hyperglycemia are particularly dangerous for patients with T1D, as they can lead to missed interventions and serious complications. Woldaregay et al. [8] highlighted that prior studies rarely account for the uneven clinical impact of such errors. To address this gap, we propose penalizing prediction errors more heavily in critical regions than in the normal range.

$$Glucose Level Regions = \{ \begin{matrix}hypoglycemia , & x < {T}_{hypo} \\ normal , & {T}_{hypo} \leq x \leq {T}_{hyper} \\ hypoglycemia , & x > {T}_{hyper}\end{matrix} \qquad ( 1 )$$

We then break down the total error, which represents the cumulative prediction error across all data points, into individual errors within each critical region. By applying specific weights to these errors, our approach ensures that the model is more sensitive to inaccuracies where they matter most, thus enhancing model reliability and effectiveness in managing T1D. This total error, which we compute as the summation of individual losses, can be formalized as shown in (2):

$${Error}_{total} = {\sum }_{t = 1}^{3} {ω}_{i} \times {Error}_{i} \qquad ( 2 )$$

The total error serves as the model's loss function aggregated over all instances, enabling it to capture and prioritize prediction accuracy within distinct regions. If we choose Mean Absolute Error (MAE) as the error term Errorᵢ, and assign weights ω_{hypo}, ωₙₒᵣₘₐₗ, and ω_{hyper} to the hypoglycemia, normal, and hyperglycemia regions, respectively, the total error can be calculated as follows:

$$M A E = \frac{1}{N} {\sum }_{i = 1}^{N} \vert {y}_{i} - {\hat{y}}_{i} \vert \qquad ( 3 )$$

$${Error}_{total} = \frac{{ω}_{h y p o}}{{n}_{1}} {\sum }_{i = 1}^{{n}_{1}} \vert {y}_{i} - {\hat{y}}_{i} \vert + \frac{{ω}_{n o r m a l}}{{n}_{2}} {\sum }_{i = 1}^{{n}_{2}} \vert {y}_{i} - {\hat{y}}_{i} \vert + \frac{{ω}_{h y p e r}}{{n}_{3}} {\sum }_{i = 1}^{{n}_{3}} \vert {y}_{i} - {\hat{y}}_{i} \vert \qquad ( 4 )$$

where n₁, n₂ and n₃ represent the total number of blood glucose samples in each region, yᵢ and ŷᵢ represent the predicted value and true value of the glucose level, respectively. The main research question that remains to be answered is how to determine the parameters of this error equation, including weight and threshold values.

### 3.3 Error Weights

We first classify CGM values into regions based on clinically defined thresholds. To emphasize dysglycemia, we fix ωₙₒᵣₘₐₗ=1 and focus on optimizing the remaining weights ω_{hypo} and ω_{hyper}. A Genetic Algorithm (GA) [35] is used to search for optimal values, offering a flexible, gradient-free approach well-suited for objectives that require training and evaluation [35-37]. This strategy ensures the loss function is tailored to patient-specific glycemic patterns and clinical priorities. The optimization procedure is detailed in Algorithm 2:

Algorithm 2 Finding the Best Pair of Weights for Each Patient

```
  1: Parameters:
  2: G ← 25 // Number of Generations
  3: N ← 20 // Population Size
  4: for each patient do
  5: Initialize population P with random weights w ∈ [1, 10]
  6: for g ← 1 to G do
  7: for each individual i ∈ P do
  8: evaluate fitness fᵢ
  9: end for
10: sort population P by fitness
11: P_{best} ← top N/2 individuals from P
12: P_{offspring} ← [ ]
13: while |P_{offspring}| < N/2 do
14: select parents p₁, p₂ ∈ P_{best}
15:  $c ← \frac{1}{2} ( p 1 + p 2 )$
16: m ← normal mutation ~ N (0, 0.5)
17: c ← clip(c + m, 1, 10)
18: P_{offspring} ← P_{offspring} ∪ c
19: end while
20: P ← P_{best} ∪ P_{offspring}
21: end for
22: ω* ← arg min(fᵢ)
23: save ω*
24: end for
```

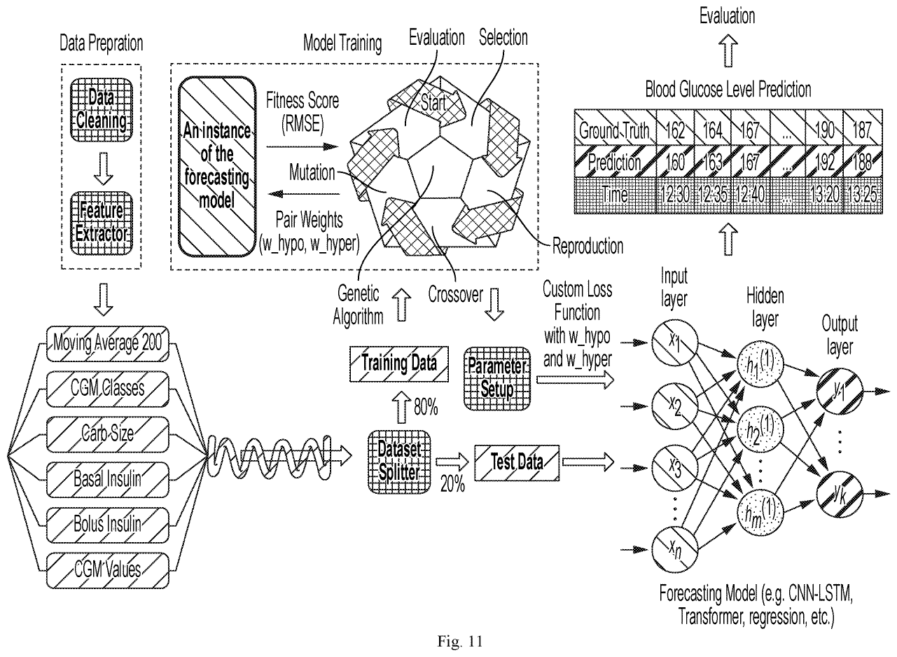

FIG. 11 shows the proposed GLIMMER methodology for predicting blood glucose levels in patients with T1D. CGM data undergo preprocessing and feature extraction before being split for training and testing. A genetic algorithm optimizes a custom loss function used to train an architecture-agnostic prediction model (e.g., CNN-LSTM, Transformer), which is then evaluated on the test data using the finalized parameters.

In summary, our methodology combines three key components to enhance prediction performance: selecting effective input features, designing a custom loss function that emphasizes clinically critical regions, and using a genetic algorithm to tune the loss function's weights. FIG. 11 illustrates the complete methodology for the design and evaluation of GLIMMER.

### 4 Data

### 4.1 OhioT1DM Dataset

We utilized the OhioT1DM dataset [38], which includes data from 12 individuals with T1D. The dataset contains raw glucose values recorded every 5 minutes, along with basal insulin, bolus insulin, and carbohydrate intake over an 8-week period.

### 4.2 AZT1D Dataset

Alongside the OhioT1DM dataset, we introduced and published a new dataset named AZT1D [26]. We collected data from 25 patients with T1D using AID systems who visited the endocrinology clinic at the Mayo Clinic in Scottsdale, AZ, between December 2023 and April 2024 for their regular appointments. The participants included 13 females and 12 males, aged between 27 and 80 years, with an average age of 59 years. Informed consent was obtained from all participants under the study protocol (IRB #23-003065). For each patient, the dataset contains an average of one month of real-world recordings, including CGM signals captured with Dexcom G6 Pro, insulin logs, meal carbohydrate sizes, and device modes (regular/sleep/exercise) recorded from the Tandem t:slim X2 insulin pump. In total, the dataset comprises 320,488 CGM entries spanning approximately 26,707 hours of monitoring data.

### 4.3 Data Preparation

The OhioT1DM dataset consists of 24 files from 12 patients, with two files per patient: one for training and one for testing [38]. To avoid overfitting and to enable hyperparameter tuning, we further split the training file chronologically, using the first 80% for training and the remaining 20% for validation. The separate testing file is kept intact and used exclusively for final evaluation. For the AZT1D dataset [26], which does not come pre-partitioned, we applied a similar strategy: each patient's data was split into 80% for training and 20% for testing, and then the training portion was again split into 80% training and 20% validation.

In addition to CGM values, basal insulin, bolus insulin, and carbohydrate amounts, we crafted two additional features. First, we computed a 200-point moving average of CGM values to capture longer-term trends and reduce sensitivity to short-term fluctuations. Second, we labeled each CGM value as hypoglycemic, normal, or hyperglycemic based on clinical thresholds (see Section 3) to help the model distinguish critical regions.

### 5 Experimental Setup

### 5.1 Architecture Configuration

To demonstrate that GLIMMER supports an architecture-agnostic design, we implemented and compared two model types: a CNN-LSTM architecture and a Transformer-based architecture. The CNN-LSTM model includes three 1D convolutional layers for local feature extraction, followed by a single LSTM layer with 8 units, reflecting configurations commonly used in prior studies [29, 39]. As an alternative, we implemented a CNN-Transformer architecture which uses the same convolutional front-end, followed by a single Transformer block with 8 attention heads, a key dimension of 16, a feed-forward size of 256, and a dropout rate of 0.1. This design enables a fair comparison of different architectural backbones under a unified training and evaluation pipeline.

### 5.2 Custom Loss Function Parameters Configuration

We performed a systematic evaluation using the genetic algorithm described in Section 3 to optimize the weights (ω_{hypo}, ω_{hyper}) in our custom loss function. The algorithm was applied to all patients in the training set, and fitness was evaluated based on RMSE on a separate validation set. The resulting weights were averaged across all patients, yielding (ω_{hypo}, ω_{hyper})=(3.296, 2.382) for the CNN-LSTM model and (4.0, 2.5) for the Transformer-based model.

### 5.3 Evaluation Metrics

To assess the performance of the GLIMMER framework, we employed standard metrics commonly used in related studies [16, 17, 22, 40], including:

### 5.3.1 Root Mean Square Error (RMSE)

RMSE quantifies the square root of the average squared differences between predicted and actual values:

$$RMSE = \sqrt{\frac{1}{n} {\sum }_{i = 1}^{n} {( {y}_{i} - {\hat{y}}_{i} )}^{2}} \qquad ( 5 )$$

where yᵢ and ŷᵢ are the actual and predicted CGM values, respectively.

### 5.3.2 Mean Absolute Error (MAE)

MAE measures the average magnitude of prediction errors:

$$M A E = \frac{1}{n} {\sum }_{i = 1}^{n} \vert {y}_{i} - {\hat{y}}_{i} \vert \qquad ( 6 )$$

### 5.3.3 Precision, Recall, and F1 Score

These metrics evaluate event-level detection (e.g., hypoglycemia onset). Precision measures the proportion of correct positive predictions, recall measures sensitivity, and Fl balances both:

$$Precision = \frac{T P}{T P + F P} , \qquad ( 7 )$$

$$Recall = \frac{T P}{T P + F N} ,$$

$$F 1 = 2 \cdot \frac{Precision \cdot Recall}{Precision + Recall}$$

### 5.3.4 Clarke Error Grid Analysis

Clarke Error Grid (CEG) [41] evaluates clinical relevance by categorizing prediction-reference pairs into five zones:
A: Accurate predictions (within 20% of reference)
B: Benign errors (no risk of inappropriate treatment)
C: Unnecessary treatment
D: Dangerous missed hypo-/hyperglycemia
E: Incorrect treatment (confusing hypo/hyper)

### 6. Results

### 6.1 GLIMMER Performance on Baseline Architectures

Table VII compares the performance of CNN-LSTM and Transformer architectures before and after applying the GLIMMER framework on the OhioT1DM and AZT1D datasets, each with a 60-minute prediction horizon (PH). Across both datasets and models, GLIMMER consistently improves performance in glucose forecasting and dysglycemia detection. On average, GLIMMER reduces RMSE by 24.6% and MAE by 29.6% for CNN-LSTM, and by 8.4% and 11.6% for Transformer. For dysglycemia classification, GLIMMER boosts recall to 98.4% and F1-score to 86.8% in the CNN-LSTM model, with similar improvements observed in the Transformer variant. These results highlight GLIMMER's ability to enhance both predictive accuracy and clinical reliability across diverse models and datasets.

Table VII shows performance comparison of CNN-LSTM and Transformer architectures before and after applying the GLIMMER framework across two datasets (PH=60 min). Metrics include RMSE and MAE for glucose prediction, and Recall, Precision, and F1 for dysglycemia.

| | | | | | |
|---|---|---|---|---|---|
| | | | Dysgly- | Dysgly- | |
| | | | cemia | cemia | Dysgly- |
| | RMSE | MAE | Recall | Precision | cemia |
| Model | (mg/dL) | (mg/dL) | (%) | (%) | F1 (%) |

OhioT1DM Dataset with PH = 60 min

| | | | | | |
|---|---|---|---|---|---|
| CNN-LSTM Baseline | 31.98 | 23.00 | 87.87 | 74.56 | 80.67 |
| GLIMMER(CNN- | 23.97 | 15.83 | 99.78 | 84.66 | 91.60 |
| LSTM) | | | | | |
| Transformer | 30.08 | 22.48 | 89.09 | 70.77 | 78.88 |
| Baseline | | | | | |
| GLIMMER(Trans- | 27.96 | 20.17 | 97.86 | 72.13 | 83.04 |
| former) | | | | | |

AZT1D Dataset with PH = 60 min

| | | | | | |
|---|---|---|---|---|---|
| CNN-LSTM Baseline | 29.55 | 21.61 | 78.33 | 45.24 | 57.35 |
| GLIMMER(CNN- | 22.48 | 15.58 | 96.97 | 71.14 | 82.07 |
| LSTM) | | | | | |
| Transformer | 28.15 | 21.27 | 79.87 | 66.11 | 72.35 |
| Baseline | | | | | |
| GLIMMER(Trans- | 25.39 | 18.50 | 89.66 | 63.17 | 74.12 |
| former) | | | | | |

Average

| | | | | | |
|---|---|---|---|---|---|
| CNN-LSTM Baseline | 30.77 | 22.31 | 83.10 | 59.90 | 69.01 |
| GLIMMER(CNN- | 23.22 | 15.71 | 98.38 | 77.90 | 86.84 |
| LSTM) | | | | | |
| Transformer | 29.12 | 21.88 | 84.48 | 68.44 | 75.62 |
| Baseline | | | | | |
| GLIMMER(Trans- | 26.68 | 19.34 | 93.76 | 67.65 | 78.58 |
| former) | | | | | |

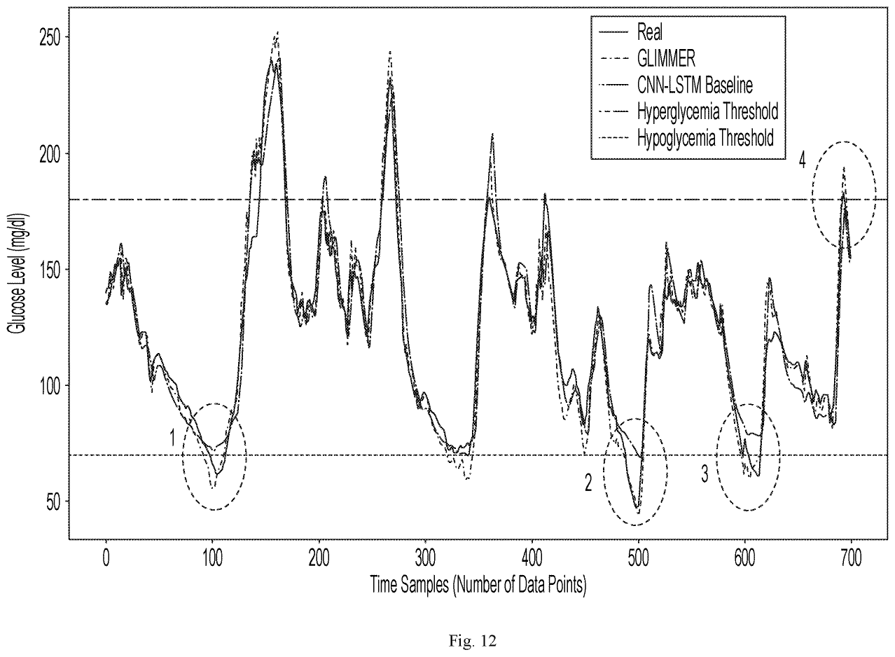

FIG. 12 shows a forecasting comparison between GLIMMER and the baseline CNN-LSTM model. The solid black line represents the real CGM values for patient 552 from the OhioT1DM test dataset. Dashed black and gray lines mark the hyperglycemia and hypoglycemia thresholds. The red circles highlight dysglycemic events that were correctly detected by GLIMMER but missed by the baseline model. This example illustrates GLIMMER's ability to identify clinically significant glycemic excursions that the baseline failed to capture.

To better illustrate the improved performance reported in Table VII, FIG. 12 presents a case study of patient 552 from the OhioT1DM dataset. In this example, GLIMMER (CNN-LSTM) successfully detects three hypoglycemia events (circled as 1, 2, and 3) and one hyperglycemia event (circle 4), while the baseline CNN-LSTM model fails to capture these critical excursions. These regions, marked by red ellipses, highlight GLIMMER's superior ability to forecast dysglycemic events and reinforce its higher recall and F1 scores in safety-critical zones.

### 6.2 Clinical Validity of GLIMMER-Enhanced Models

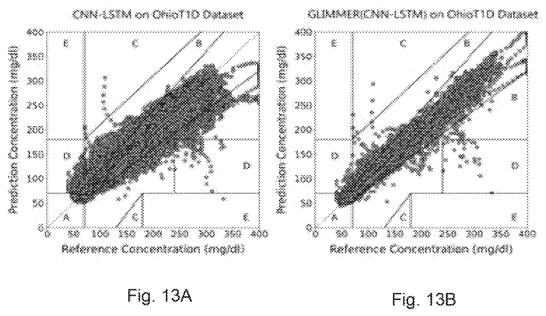

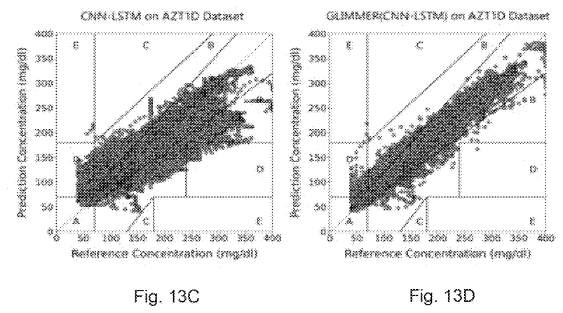

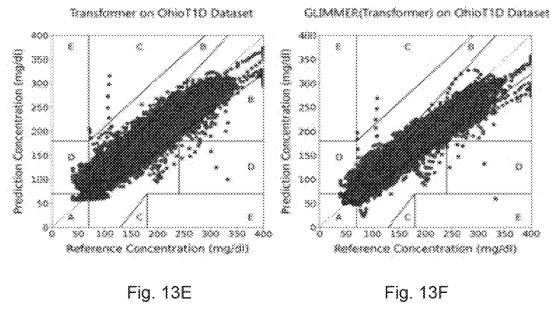

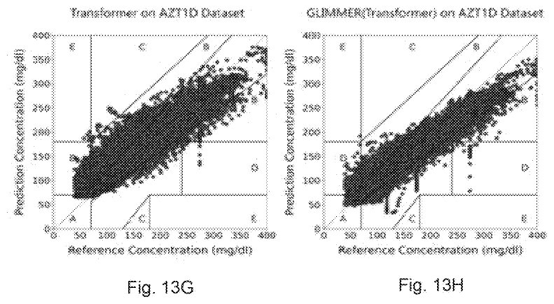

To further assess the clinical reliability of glucose predictions, we conducted a CEG analysis, which classifies prediction errors into zones based on their potential clinical consequences. As shown in Table VIII and visualized in FIG. 13, GLIMMER consistently increases the proportion of predictions falling within Zone A—representing clinically accurate and safe predictions—while reducing the presence of errors in critical-risk zones (D and E).

Table VIII shows CEG zone distribution (%) for glucose predictions made by CNN-LSTM and Transformer architectures, before and after applying GLIMMER, on the OhioT1DM and AZT1D datasets (PH=60 minutes). Zone A reflects clinically accurate predictions, while Zones D and E indicate potentially dangerous errors.

| | | | | | |
|---|---|---|---|---|---|
| Model | Zone A | Zone B | Zone C | Zone D | Zone E |

OhioT1DM Dataset with PH = 60 min

| | | | | | |
|---|---|---|---|---|---|
| CNN-LSTM Baseline | 74.31 | 23.12 | 0.11 | 2.43 | 0.03 |
| GLIMMER(CNN- | 85.46 | 13.26 | 0.13 | 1.12 | 0.02 |
| LSTM) | | | | | |
| Transformer | 72.78 | 24.08 | 0.14 | 2.99 | 0.00 |
| Baseline | | | | | |
| GLIMMER(Transformer) | 79.67 | 18.50 | 0.07 | 1.72 | 0.04 |

AZT1D Dataset with PH = 60 min

| | | | | | |
|---|---|---|---|---|---|
| CNN-LSTM Baseline | 73.27 | 24.47 | 0.03 | 2.21 | 0.02 |
| GLIMMER(CNN- | 83.89 | 14.94 | 0.02 | 1.12 | 0.02 |
| LSTM) | | | | | |
| Transformer | 73.01 | 24.92 | 0.04 | 2.00 | 0.02 |
| Baseline | | | | | |
| GLIMMER(Transformer) | 80.26 | 18.17 | 0.01 | 1.54 | 0.02 |

Average

| | | | | | |
|---|---|---|---|---|---|
| CNN-LSTM Baseline | 73.79 | 23.80 | 0.07 | 2.32 | 0.03 |
| GLIMMER(CNN- | 84.68 | 14.10 | 0.08 | 1.12 | 0.02 |
| LSTM) | | | | | |
| Transformer | 72.90 | 24.50 | 0.09 | 2.50 | 0.01 |
| Baseline | | | | | |
| GLIMMER(Transformer) | 79.97 | 18.34 | 0.04 | 1.63 | 0.03 |

In the OhioT1DM dataset, GLIMMER improves Zone A coverage from 74.3% to 85.5% for CNN-LSTM, and from 72.8% to 79.7% for Transformer, with corresponding reductions in Zones D and E. In AZT1D, Zone A rises from 73.3% to 83.9% for CNN-LSTM and from 73.0% to 80.3% for Transformer, with Zone D errors nearly halved or more.

Averaged across datasets, CNN-LSTM with GLIMMER achieves a 10.9 percentage point gain in Zone A and a reduction of over 50% in Zone D errors. Transformer shows a 7-point improvement in Zone A and similarly low rates of high-risk misclassifications. These results demonstrate that GLIMMER not only enhances dysglycemia detection and predictive accuracy, but also significantly improves clinical safety by reducing the likelihood of dangerous prediction errors.

FIGS. 13A-13H shows Clarke Error Grid for CNN-LSTM and Transformer models, with and without GLIMMER, across both datasets. FIGS. 13A-13D correspond to the OhioT1DM and AZT1D datasets using the CNN-LSTM baseline model and its GLIMMER-enhanced version, respectively. FIGS. 13E-13H show the same comparison using the Transformer architecture. In each case, GLIMMER improves the clustering of predictions near the x=y line.

### 6.3 Benchmarking Model Performance and Complexity

Table IX benchmarks various glucose forecasting models on the OhioT1DM dataset using RMSE, parameter count, and RMSE-based ranking. While top-performing models such as TimesNet [20], BG-BERT [25], and Gluformer [24] achieve strong predictive accuracy, they require a substantial number of parameters—ranging from 2 million to nearly 19 million—and typically depend on powerful GPU hardware for training and inference. In contrast, GLIMMER(CNN-LSTM) achieves a competitive RMSE of 23.97 mg/dL and ranks third overall, while using only 10,000 parameters—over 1,800× fewer than TimesNet and 200× fewer than BG-BERT. This result highlights GLIMMER's ability to approach the performance of large transformer-based models with drastically lower computational cost, making it far more suitable for real-time deployment in mobile or resource-constrained clinical environments. By optimizing baseline architectures through the GLIMMER framework, we demonstrate that it is possible to achieve both high predictive performance and lightweight design—an essential combination for integration into diabetes management tools such as AID systems.

Table IX shows a comparison of RMSE and parameter count of glucose forecasting models on the OhioT1DM dataset (PH=60 minutes).

| Model | RMSE (mg/dL) | Params |
|---|---|---|
| FCNN [22] | 31.07 | — |
| CRNN [16] | 32.02 | — |
| Bi-LSTM [18] | 33.44 | — |
| Informer [19] | 25.76 | 181k |
| TimesNet [20] | 23.48 | 18,749k |
| Gluformer [24] | 27.14 | 11,247k |
| BG-BERT [25] | 23.67 | 2,091k |
| CNN-LSTM Baseline | 31.98 | 10k |
| GLIMMER(CNN-LSTM) | 23.97 | 10k |
| Transformer Baseline | 30.08 | 50k |
| GLIMMER(Transformer) | 27.96 | 50k |

### 7 Discussion

The results demonstrate that the GLIMMER framework consistently improves the predictive performance of baseline architectures, achieving accuracy comparable to state-of-the-art models while maintaining a lightweight design. Unlike many high-performing models that require millions of parameters and complex inputs, GLIMMER-enhanced models achieve competitive RMSE scores with drastically fewer parameters, enabling deployment on mobile or embedded devices. Moreover, CEG analysis confirms the clinical reliability of GLIMMER, with notable gains in Zone A predictions and reduced dangerous errors.

In contrast to prior works like Cichosz et al. [42], which applied static penalties to prioritize clinical regions, GLIMMER incorporates a data-driven loss function that adaptively learns importance weights using a genetic algorithm. This allows more flexible prioritization of critical prediction errors based on dataset characteristics and clinical goals. GLIMMER's modular design also supports plug-and-play integration with different model architectures, as shown by its successful application to both CNN-LSTM and Transformer backbones.

Despite these advantages, limitations remain. GLIMMER's performance in hypoglycemia detection is constrained by the limited number of such events in the training data. This sparsity reduces the model's ability to generalize to low-glucose conditions, a challenge shared across many datasets in this domain. Future work may benefit from data augmentation strategies or curated sampling techniques to address this imbalance.

Looking ahead, we aim to extend the prediction horizon to 120 minutes, which introduces new challenges due to increased uncertainty over time. To address this, we plan to incorporate longer-term features—such as aggregated physiological trends and behavioral patterns—that may improve predictive accuracy across extended intervals. Additionally, we intend to expand the AZT1D dataset from 25 to 100 patients. A larger and more diverse cohort will enhance model generalizability across patient populations and support the development of more robust and personalized forecasting tools. A potential direction for future investigation is the joint optimization of glycemic thresholds and loss function weights, allowing the GLIMMER framework to better adapt to individual treatment goals and physiological variability.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The glucose level indicator model with modified error rate can be a component of a single system, multiple systems, and/or geographically separate systems. The glucose level indicator model with modified error rate can also be a subcomponent or subsystem of a single system, multiple systems, and/or geographically separate systems. The components of glucose level indicator model with modified error rate can be coupled to one or more other components (not shown) of a host system or a system coupled to the host system.

One or more components of the glucose level indicator model with modified error rate and/or a corresponding interface, system or application to which the glucose level indicator model with modified error rate is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the glucose level indicator model with modified error rate can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the glucose level indicator model with modified error rate and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the glucose level indicator model with modified error rate and corresponding systems and methods include: microcontrollers with memory (such as electronically erasable programmable read only memory (EEPROM)), embedded microprocessors, firmware, software, etc. Furthermore, aspects of the glucose level indicator model with modified error rate and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the glucose level indicator model with modified error rate is not intended to be exhaustive or to limit the systems and methods to the precise forms disclosed. While specific embodiments of, and examples for, the glucose level indicator model with modified error rate and corresponding systems and methods are described herein for illustrative purposes, various equivalent modifications are possible within the scope of the systems and methods, as those skilled in the relevant art will recognize. The teachings of the glucose level indicator model with modified error rate and corresponding systems and methods provided herein can be applied to other systems and methods, not only for the systems and methods described above.

The elements and acts of the various embodiments described above can be combined to provide further embodiments. These and other changes can be made to the glucose level indicator model with modified error rate and corresponding systems and methods in light of the above detailed description.

### EXAMPLE 1 REFERENCES

[1] M. A. Atkinson, G. S. Eisenbarth, and A. W. Michels, “Type 1 diabetes,” The lancet, vol. 383, no. 9911, pp. 69-82, 2014.
[2] D. Control, C. T. of Diabetes Interventions, and C. D. S. R. Group, “Intensive diabetes treatment and cardiovascular disease in patients with type 1 diabetes,” New England Journal of Medicine, vol. 353, no. 25, pp. 2643-2653, 2005.
[3] M. Karvonen, M. Viik-Kajander, E. Moltchanova, I. Libman, R. LaPorte, and J. Tuomilehto, “Incidence of childhood type 1 diabetes worldwide. diabetes mondiale (diamond) project group.” Diabetes care, vol. 23, no. 10, pp. 1516-1526, 2000.
[4] S. J. Guo and H. Shao, “Growing global burden of type 1 diabetes needs multitiered precision public health interventions,” The Lancet Diabetes & Endocrinology, vol. 10, no. 10, pp. 688-689, 2022.
[5] L. Pan, Q. Mukhtar, and L. Geiss, “Self-monitoring of blood glucose among adults with diabetes-united states, 1997-2006.” MMWR: Morbidity & Mortality Weekly Report, vol. 56, no. 43, 2007.
[6] A. D. Association, “5. glycemic targets,” Diabetes care, vol. 39, no. Supplement 1, pp. S39-S46, 2016.
[7] S. Knapp, P. Manroa, and K. Doshi, “Self-monitoring of blood glucose: Advice for providers and patients.” Cleveland Clinic Journal of Medicine, vol. 83, no. 5, pp. 355-360, 2016.
[8] D. Care, “6. glycemic targets: standards of medical care in diabetes—2019,” Diabetes Care, vol. 42, no. Supplement 1, pp. S61-70, 2019.
[9] D. DeSalvo and B. Buckingham, “Continuous glucose monitoring: current use and future directions,” Current diabetes reports, vol. 13, pp. 657-662, 2013.
[10] J. K. Nielsen, C. B. Djurhuus, C. H. Gravholt, A. C. Carus, J. Granild-Jensen, H. Ørskov, and J. S. Christiansen, “Continuous glucose monitoring in interstitial subcutaneous adipose tissue and skeletal muscle reflects excursions in cerebral cortex,” Diabetes, vol. 54, no. 6, pp. 1635-1639, 2005.
[11] A. Basu, S. Dube, S. Veettil, M. Slama, Y. C. Kudva, T. Peyser, R. E. Carter, C. Cobelli, and R. Basu, “Time lag of glucose from intravascular to interstitial compartment in type 1 diabetes,” Journal of diabetes science and technology, vol. 9, no. 1, pp. 63-68, 2014.
[12] S. Templer, “Closed-loop insulin delivery systems: past, present, and future directions,” Frontiers in Endocrinology, vol. 13, p. 919942, 2022.
[13] M. Tauschmann, H. Thabit, L. Bally, J. M. Allen, S. Hartnell, M. E. Wilinska, Y. Ruan, J. Sibayan, C. Kollman, P. Cheng et al., “Closed-loop insulin delivery in suboptimally controlled type 1 diabetes: a multicentre, 12-week randomised trial,” The Lancet, vol. 392, no. 10155, pp. 1321-1329, 2018.
[14] J. L. Sherr, L. Heinemann, G. A. Fleming, R. M. Bergenstal, D. Bruttomesso, H. Hanaire, R. W. Holl, J. R. Petrie, A. L. Peters, and M. Evans, “Automated insulin delivery: benefits, challenges, and recommendations. a consensus report of the joint diabetes technology working group of the european association for the study of diabetes and the american diabetes association,” Diabetes Care, vol. 45, no. 12, pp. 3058-3074, 2022.
[15] E. Renard, M. Joubert, O. Villard, B. Dreves, Y. Reznik, A. Farret, J. Place, M. D. Breton, B. P. Kovatchev, and iDCL Trial Research Group, “Safety and efficacy of sustained automated insulin delivery compared with sensor and pump therapy in adults with type 1 diabetes at high risk for hypoglycemia: a randomized controlled trial,” Diabetes Care, vol. 46, no. 12, pp. 2180-2187, 2023.
[16] C. Limbert, A. J. Kowalski, and T. P. Danne, “Automated insulin delivery: A milestone on the road to insulin independence in type 1 diabetes,” Diabetes Care, vol. 47, no. 6, pp. 918-920, 2024.
[17] A. Chico, J. Moreno-Fernindez, D. Fernindez-Garcia, and E. Soli, “The hybrid closed-loop system tandem t: slim x2™ with controliq technology: expert recommendations for better management and optimization,” Diabetes Therapy, vol. 15, no. 1, pp. 281-295, 2024.
[18] J. W. Lum, R. J. Bailey, V. Barnes-Lomen, D. Naranjo, K. K. Hood, R. A. Lal, B. Arbiter, A. S. Brown, D. J. DeSalvo, J. Pettus et al., “A real-world prospective study of the safety and effectiveness of the loop open source automated insulin delivery system,” Diabetes technology & therapeutics, vol. 23, no. 5, pp. 367-375, 2021.
[19] R. W. Beck, L. G. Kanapka, M. D. Breton, S. A. Brown, R. P. Wadwa, B. A. Buckingham, C. Kollman, and B. Kovatchev, “A meta-analysis of randomized trial outcomes for the t: slim x2 insulin pump with control-iq technology in youth and adults from age 2 to 72,” Diabetes Technology & Therapeutics, vol. 25, no. 5, pp. 329-342, 2023.
[20] S. Oviedo, J. Vehí, R. Calm, and J. Armengol, “A review of personalized blood glucose prediction strategies for t1dm patients,” International journal for numerical methods in biomedical engineering, vol. 33, no. 6, p. e2833, 2017.
[21] T. Zhu, K. Li, P. Herrero, J. Chen, and P. Georgiou, “A deep learning algorithm for personalized blood glucose prediction.” in KDH@IJCAI, 2018, pp. 64-78.
[22] J. L. Parkes, S. L. Slatin, S. Pardo, and B. H. Ginsberg, “A new consensus error grid to evaluate the clinical significance of inaccuracies in the measurement of blood glucose.” Diabetes care, vol. 23, no. 8, pp. 1143-1148, 2000.
[23] R. E. Pratley, L. G. Kanapka, M. R. Rickels, A. Ahmann, G. Aleppo, R. Beck, A. Bhargava, B. W. Bode, A. Carlson, N. S. Chaytor et al., “Effect of continuous glucose monitoring on hypoglycemia in older adults with type 1 diabetes: a randomized clinical trial,” Jama, vol. 323, no. 23, pp. 2397-2406, 2020.
[24] M. Marigliano, C. Piona, V. Mancioppi, E. Morotti, A. Morandi, and C. Maffeis, “Glucose sensor with predictive alarm for hypoglycaemia: Improved glycaemic control in adolescents with type 1 diabetes,” Diabetes, Obesity and Metabolism, vol. 26, no. 4, pp. 1314-1320, 2024.
[25] M. Vettoretti, G. Cappon, A. Facchinetti, and G. Sparacino, “Advanced diabetes management using artificial intelligence and continuous glucose monitoring sensors,” Sensors, vol. 20, no. 14, p. 3870, 2020.
[26] P. Shroff, A. Arefeen, and H. Ghasemzadeh, “Glucoseassist: Personalized blood glucose level predictions and early dysglycemia detection,” in 2023 IEEE 19th International Conference on Body Sensor Networks (BSN). IEEE, 2023, pp. 1-4.
[27] A. Arefeen, S. Fessler, C. Johnston, and H. Ghasemzadeh, “Forewarning postprandial hyperglycemia with interpretations using machine learning,” in 2022 IEEE-EMBS International Conference on Wearable and Implantable Body Sensor Networks (BSN). IEEE, 2022, pp. 1-4.
[28] T. Zhu, K. Li, P. Herrero, and P. Georgiou, “Personalized blood glucose prediction for type 1 diabetes using evidential deep learning and metalearning,” IEEE Transactions on Biomedical Engineering, vol. 70, no. 1, pp. 193-204, 2022.
[29] G. Keren and B. Schuller, “Convolutional rnn: an enhanced model for extracting features from sequential data,” in 2016 International Joint Conference on Neural Networks 15 (IJCNN). IEEE, 2016, pp. 3412-3419.
[30] K. Li, J. Daniels, C. Liu, P. Herrero, and P. Georgiou, “Convolutional recurrent neural networks for glucose prediction,” IEEE journal of biomedical and health informatics, vol. 24, no. 2, pp. 603-613, 2019.
[31] Z. Li, F. Liu, W. Yang, S. Peng, and J. Zhou, “A survey of convolutional neural networks: analysis, applications, and prospects,” IEEE transactions on neural networks and learning systems, vol. 33, no. 12, pp. 6999-7019, 2021.
[32] L. R. Medsker, L. Jain et al., “Recurrent neural networks,” Design and Applications, vol. 5, no. 64-67, p. 2, 2001.
[33] S. Hochreiter and J. Schmidhuber, “Long short-term memory,” Neural computation, vol. 9, no. 8, pp. 1735-1780, 1997.
[34] A. Aliberti, I. Pupillo, S. Terna, E. Macii, S. Di Cataldo, E. Patti, and A. Acquaviva, “A multi-patient data-driven approach to blood glucose prediction,” IEEE Access, vol. 7, pp. 69 311-69 325, 2019.
[35] S. A. Billings, Nonlinear system identification: NARMAX methods in the time, frequency, and spatio-temporal domains. John Wiley & Sons, 2013.
[36] Q. Sun, M. V. Jankovic, L. Bally, and S. G. Mougiakakou, “Predicting blood glucose with an lstm and bi-lstm based deep neural network,” in 2018 14th symposium on neural networks and applications (NEUREL). IEEE, 2018, pp. 1-5.
[37] H. Butt, I. Khosa, and M. A. Iftikhar, “Feature transformation for efficient blood glucose prediction in type 1 diabetes mellitus patients,” Diagnostics, vol. 13, no. 3, p. 340, 2023.
[38] M. Zhang, K. B. Flores, and H. T. Tran, “Deep learning and regression approaches to forecasting blood glucose levels for type 1 diabetes,” Biomedical Signal Processing and Control, vol. 69, p. 102923, 2021.
[39] W. Lu, J. Lᵢ, Y. Lᵢ, A. Sun, and J. Wang, “A cnn-lstm-based model to forecast stock prices,” Complexity, vol. 2020, no. 1, p. 6622927, 2020.
[40] M. Alhussein, K. Aurangzeb, and S. I. Haider, “Hybrid cnn-lstm model for short-term individual household load forecasting,” Ieee Access, vol. 8, pp. 180 544-180 557, 2020.
[41] Q. Wu, F. Guan, C. Lv, and Y. Huang, “Ultra-short-term multi-step wind power forecasting based on cnn-lstm,” IET Renewable Power Generation, vol. 15, no. 5, pp. 1019-1029, 2021.
[42] M. Jaloli and M. Cescon, “Long-term prediction of blood glucose levels in type 1 diabetes using a cnn-lstm-based deep neural network,” Journal of diabetes science and technology, vol. 17, no. 6, pp. 1590-1601, 2023.
[43] N. Srivastava, G. Hinton, A. Krizhevsky, I. Sutskever, and R. Salakhutdinov, 20 “Dropout: a simple way to prevent neural networks from overfitting,” The journal of machine learning research, vol. 15, no. 1, pp. 1929-1958, 2014.
[44] A. Z. Woldaregay, E. Arsand, S. Walderhaug, D. Albers, L. Mamykina, T. Botsis, and G. Hartvigsen, “Data-driven modeling and prediction of blood glucose dynamics: Machine learning applications in type 1 diabetes,” Artificial intelligence in medicine, vol. 98, pp. 109-134, 2019.
[45] M. Mitchell, An introduction to genetic algorithms. MIT press, 1998.
[46] Y. Jin, “A comprehensive survey of fitness approximation in evolutionary computation,” Soft computing, vol. 9, no. 1, pp. 3-12, 2005.
[47] S. Khamesian and H. Malek, “Hybrid self-attention neat: a novel evolutionary self-attention approach to improve the neat algorithm in high dimensional inputs,” Evolving Systems, vol. 15, no. 2, pp. 489-503, 2024.
[48] C. Marling and R. Bunescu, “The ohiot1dm dataset for blood glucose level prediction: Update 2020,” in CEUR workshop proceedings, vol. 2675. NIH Public Access, 2020, p. 71.
[49] S. Mirshekarian, H. Shen, R. Bunescu, and C. Marling, “Lstms and neural attention models for blood glucose prediction: Comparative experiments on real and synthetic data,” in 2019 41st annual international conference of the IEEE engineering in medicine and biology society (EMBC). IEEE, 2019, pp. 706-712.
[50] A. Zheng and A. Casari, Feature engineering for machine learning: principles and techniques for data scientists. “O'Reilly Media, Inc.”, 2018.
[51] P. J. Brockwell and R. A. Davis, Introduction to time series and forecasting. Springer, 2002.
[52] T. El Idrissi and A. Idri, “Deep learning for blood glucose prediction: Cnn vs lstm,” in Computational Science and Its Applications—ICCSA 2020: 20th International Conference, Cagliari, Italy, Jul. 1-4, 2020, Proceedings, Part II 20. Springer, 2020, pp. 379-393.
[53] J. J. Dylag, “Machine learning based prediction of glucose levels in type 1 diabetes patients with the use of continuous glucose monitoring data,” arXiv preprint arXiv:2302.12856, 2023.
[54] W. L. Clarke, D. Cox, L. A. Gonder-Frederick, W. Carter, and S. L. Pohl, “Evaluating clinical accuracy of systems for self-monitoring of blood glucose,” Diabetes care, vol. 10, no. 5, pp. 622-628, 1987.
[55] L. Yang, T. L. J. Ng, B. Smyth, and R. Dong, “Html: Hierarchical transformer-based multi-task learning for volatility prediction,” in Proceedings of The Web Conference 2020, 2020, pp. 441-451.
[56] E. I. Georga, V. C. Protopappas, D. Ardigo, M. Marina, I. Zavaroni, D. Polyzos, and D. I. Fotiadis, “Multivariate prediction of subcutaneous glucose concentration in type 1 diabetes patients based on support vector regression,” IEEE journal of biomedical and health informatics, vol. 17, no. 1, pp. 71-81, 2012.
[57] E. I. Georga, V. C. Protopappas, D. Polyzos, and D. I. Fotiadis, “A predictive model of subcutaneous glucose concentration in type 1 diabetes based on random forests,” in 2012 Annual International Conference of the IEEE Engineering in Medicine and Biology Society. IEEE, 2012, pp. 2889-2892.
[58] K. Plis, R. Bunescu, C. Marling, J. Shubrook, and F. Schwartz, “A machine learning approach to predicting blood glucose levels for diabetes management,” in Workshops at the Twenty-Eighth AAAI conference on artificial intelligence, 2014.
[59] G. Annuzzi, A. Apicella, P. Arpaia, L. Bozzetto, S. Criscuolo, E. De Benedetto, M.

Pesola, and R. Prevete, “Exploring nutritional influence on blood glucose forecasting for type 1 diabetes using explainable ai,” IEEE journal of biomedical and health informatics, 2023.

### EXAMPLE 2 REFERENCES

[1] Guo, S. J., Shao, H.: Growing global burden of type 1 diabetes needs multitiered precision public health interventions. The Lancet Diabetes & Endocrinology 10(10), 688-689 (2022)
[2] Atkinson, M. A., Eisenbarth, G. S., Michels, A. W.: Type 1 diabetes. The lancet 383(9911), 69-82 (2014)
[3] Control, D., Diabetes Interventions, C.T., Group, C.D.S.R.: Intensive diabetes treatment and cardiovascular disease in patients with type 1 diabetes. New England Journal of Medicine 353(25), 2643-2653 (2005)
[4] Karvonen, M., Viik-Kajander, M., Moltchanova, E., Libman, I., LaPorte, R., Tuomilehto, J.: Incidence of childhood type 1 diabetes worldwide. diabetes mondiale (diamond) project group. Diabetes care 23(10), 1516-1526 (2000)
[5] Shroff, P., Arefeen, A., Ghasemzadeh, H.: Glucoseassist: Personalized blood glucose level predictions and early dysglycemia detection. In: 2023 IEEE 19th International Conference on Body Sensor Networks (BSN), pp. 1-4 (2023). IEEE
[6] Della Cioppa, A., De Falco, I., Koutny, T., Scafuri, U., Ubl, M., Tarantino, E.: Reducing high-risk glucose forecasting errors by evolving interpretable models for type 1 diabetes. Applied Soft Computing 134, 110012 (2023)
[7] Nemat, H., Khadem, H., Elliott, J., Benaissa, M.: Data-driven blood glucose level prediction in type 1 diabetes: a comprehensive comparative analysis. Scientific reports 14(1), 21863 (2024)
[8] Woldaregay, A. Z., ° Arsand, E., Walderhaug, S., Albers, D., Mamykina, L., Botsis, T., Hartvigsen, G.: Data-driven modeling and prediction of blood glucose dynamics: Machine learning applications in type 1 diabetes. Artificial intelligence in medicine 98, 109-134 (2019)
[9] Marigliano, M., Piona, C., Mancioppi, V., Morotti, E., Morandi, A., Maffeis, C.: Glucose sensor with predictive alarm for hypoglycaemia: Improved glycaemic control in adolescents with type 1 diabetes. Diabetes, Obesity and Metabolism 26(4), 1314-1320 (2024)
[10] Battelino, T., Danne, T., Bergenstal, R. M., Amiel, S. A., Beck, R., Biester, T., Bosi, E., Buckingham, B. A., Cefalu, W. T., Close, K. L., et al.: Clinical targets for continuous glucose monitoring data interpretation: recommendations from the international consensus on time in range. Diabetes care 42(8), 1593-1603 (2019)
[11] DeSalvo, D., Buckingham, B.: Continuous glucose monitoring: current use and future directions. Current diabetes reports 13, 657-662 (2013)
[12] Limbert, C., Kowalski, A. J., Danne, T. P.: Automated insulin delivery: A milestone on the road to insulin independence in type 1 diabetes. Diabetes Care 47(6), 918-920 (2024)
[13] Templer, S.: Closed-loop insulin delivery systems: past, present, and future directions.

Frontiers in Endocrinology 13, 919942 (2022)
[14] Sherr, J. L., Heinemann, L., Fleming, G. A., Bergenstal, R. M., Bruttomesso, D., Hanaire, H., Holl, R. W., Petrie, J. R., Peters, A. L., Evans, M.: Automated insulin delivery: benefits, challenges, and recommendations. a consensus report of the joint diabetes technology working group of the european association for the study of diabetes and the american diabetes association. Diabetes Care 45(12), 3058-3074 (2022)
[15] Renard, E., Joubert, M., Villard, O., Dreves, B., Reznik, Y., Farret, A., Place, J., Breton, M. D., Kovatchev, B. P., Trial Research Group: Safety and efficacy of sustained automated insulin delivery compared with sensor and pump therapy in adults with type 1 diabetes at high risk for hypoglycemia: a randomized controlled trial. Diabetes Care 46(12), 2180-2187 (2023)
[16] Lᵢ, K., Daniels, J., Liu, C., Herrero, P., Georgiou, P.: Convolutional recurrent neural networks for glucose prediction. IEEE journal of biomedical and health informatics 24(2), 603-613 (2019)
[17] Aliberti, A., Pupillo, I., Terna, S., Macii, E., Di Cataldo, S., Patti, E., Acquaviva, A.: A multi-patient data-driven approach to blood glucose prediction. IEEE Access 7, 69311-69325 (2019)
[18] Sun, Q., Jankovic, M. V., Bally, L., Mougiakakou, S. G.: Predicting blood glucose with an lstm and bi-lstm based deep neural network. In: 2018 14th Symposium on Neural Networks and Applications (NEUREL), pp. 1-5 (2018). IEEE
[19] Zhou, H., Zhang, S., Peng, J., Zhang, S., Lᵢ, J., Xiong, H., Zhang, W.: Informer: Beyond efficient transformer for long sequence time-series forecasting. In: Proceedings of the AAAI Conference on Artificial Intelligence, vol. 35, pp. 11106-11115 (2021)
[20] Wu, H., Hu, T., Liu, Y., Zhou, H., Wang, J., Long, M.: Timesnet: Temporal 2d-variation modeling for general time series analysis. arXiv preprint arXiv:2210.02186 (2022)
[21] Del Giudice, L. L., Piersanti, A., Göbl, C., Burattini, L., Tura, A., Morettini, M.: Availability of open dynamic glycemic data in the field of diabetes research: A scoping review. Journal of Diabetes Science and Technology, 19322968251316896 (2025)
[22] Zhu, T., Li, K., Herrero, P., Georgiou, P.: Personalized blood glucose prediction for type 1 diabetes using evidential deep learning and meta-learning. IEEE Transactions on Biomedical Engineering 70(1), 193-204 (2022)
[23] Zhang, M., Flores, K. B., Tran, H. T.: Deep learning and regression approaches to forecasting blood glucose levels for type 1 diabetes. Biomedical Signal Processing and Control 69, 102923 (2021)
[24] Sergazinov, R., Armandpour, M., Gaynanova, I.: Gluformer: Transformer-based personalized glucose forecasting with uncertainty quantification. In: ICASSP 2023-2023 IEEE International Conference on Acoustics, Speech and Signal Processing (ICASSP), pp. 1-5 (2023). IEEE
[25] Zheng, X., Ji, S., Wu, C.: Predicting adverse events for patients with type-1 diabetes via self-supervised learning. In: ICASSP 2024-2024 IEEE International Conference on Acoustics, Speech and Signal Processing (ICASSP), pp. 1526-1530 (2024). IEEE
[26] Khamesian, S., Arefeen, A., Thompson, B. M., Grando, M. A., Ghasemzadeh, H.: Azt1d: A real-world dataset for type 1 diabetes. arXiv preprint arXiv:2506.14789 (2025)
[27] Vettoretti, M., Cappon, G., Facchinetti, A., Sparacino, G.: Advanced diabetes management using artificial intelligence and continuous glucose monitoring sensors. Sensors 20(14), 3870(2020)
[28] Arefeen, A., Fessler, S., Johnston, C., Ghasemzadeh, H.: Forewarning post-prandial hyperglycemia with interpretations using machine learning. In: 2022 IEEE-EMBS International Conference on Wearable and Implantable Body Sensor Networks (BSN), pp. 1-4 (2022). IEEE
[29] El Idrissi, T., Idri, A.: Deep learning for blood glucose prediction: Cnn vs lstm. In: Computational Science and Its Applications-ICCSA 2020: 20th International Conference, Cagliari, Italy, Jul. 1-4, 2020, Proceedings, Part II 20, pp. 379-393 (2020). Springer
[30] Lu, W., Li, J., Li, Y., Sun, A., Wang, J.: A cnn-lstm-based model to forecast stock prices.

Complexity 2020(1), 6622927 (2020)
[31] Plis, K., Bunescu, R. C., Marling, C., Shubrook, J., Schwartz, F.: A machine learning approach to predicting blood glucose levels for diabetes management. In: AAAI Workshop: Modern Artificial Intelligence for Health Analytics, pp. 35-39 (2014)
[32] Georga, E. I., Protopappas, V. C., Ardigo, D., Marina, M., Zavaroni, I., Polyzos, D., Fotiadis, D. I.: Multivariate prediction of subcutaneous glucose concentration in type 1 diabetes patients based on support vector regression. IEEE journal of biomedical and health informatics 17(1), 71-81 (2012)
[33] Jaloli, M., Cescon, M.: Long-term prediction of blood glucose levels in type 1 diabetes using a cnn-lstm-based deep neural network. Journal of diabetes science and technology 17(6), 1590-1601 (2023)
[34] Chico, A., Moreno-Fernindez, J., Fernindez-Garcia, D., Soli, E.: The hybrid closed-loop system tandem t: slim x2™ with control-iq technology: expert recommendations for better management and optimization. Diabetes Therapy 15(1), 281-295 (2024)
[35] Immanuel, S. D., Chakraborty, U. K.: Genetic algorithm: An approach on optimization. In: 2019 International Conference on Communication and Electronics Systems (ICCES), pp. 701-708 (2019). IEEE
[36] Jin, Y.: A comprehensive survey of fitness approximation in evolutionary computation. Soft computing 9(1), 3-12 (2005)
[37] Khamesian, S., Malek, H.: Hybrid self-attention neat: a novel evolutionary self-attention approach to improve the neat algorithm in high dimensional inputs. Evolving Systems 15(2), 489-503 (2024)
[38] Marling, C., Bunescu, R.: The ohiot1dm dataset for blood glucose level prediction: Update 2020. In: CEUR Workshop Proceedings, vol. 2675, p. 71 (2020). NIH Public Access
[39] Dylag, J. J.: Machine learning based prediction of glucose levels in type 1 diabetes patients with the use of continuous glucose monitoring data. arXiv preprint arXiv:2302.12856 (2023)
[40] Butt, H., Khosa, I., Iftikhar, M. A.: Feature transformation for efficient blood glucose prediction in type 1 diabetes mellitus patients. Diagnostics 13(3), 340 (2023)
[41] Clarke, W. L., Cox, D., Gonder-Frederick, L. A., Carter, W., Pohl, S. L.: Evaluating clinical accuracy of systems for self-monitoring of blood glucose. Diabetes care 10(5), 622-628 (1987)
[42] Cichosz, S. L., Kronborg, T., Jensen, M. H., Hejlesen, O.: Penalty weighted glucose prediction models could lead to better clinically usage. Computers in Biology and Medicine 138, 104865 (2021)

1. A system comprising,
at least one application running on one or more processors of a server, wherein the at least one application is communicatively coupled with one or more sensor devices and a remote mobile device, the one or more sensor devices configured to monitor real time biometric data of a subject;
the one or more sensing devices configured to transmit the real time biometric data to the at least one application, the at least one application configured to:
extract feature data variables from the real time biometric data,
apply a predictive model to the feature data variables to predict glucose levels of the subject over a future period of time, and
communicate a signal to the mobile device when predicted glucose levels cross either an upper or lower threshold.

2. The system of claim 1, wherein the one or more sensor devices comprises a t:Slim X2™ insulin pump.

3. The system of claim 1, wherein the one or more devices comprises a Dexcom G6™ interstitial glucose sensor.

4. The system of claim 1, wherein the feature data variables comprise an amount of basal insulin delivered to the subject every hour.

5. The system of claim 1, wherein the feature data variables comprise an amount of carbohydrates in grams consumed by the subject.

6. The system of claim 1, wherein the feature data variables comprise an amount of bolus insulin scheduled for release.

7. The system of claim 1, wherein the feature data variables comprise raw continuous glucose levels.

8. The system of claim 1, wherein the feature data variables comprise moving average glucose levels.

9. The system of claim 1, wherein the feature data variables comprise glucose level classifications.

10. The system of claim 9, wherein a first glucose level classification comprises a glucose level below a first threshold.

11. The system of claim 10, wherein a second glucose level classification comprises a glucose level equal to or above the first threshold and equal to or below a second threshold.

12. The system of claim 11, wherein a third glucose level classification comprises a glucose level above the second threshold.

13. The system of claim 12, wherein the predictive model comprises a CNN-LSTM model, wherein a loss function of the CNN-LSTM model comprises a mean square error function, wherein the mean square errors are weighted according to the glucose level classifications, wherein output of the mean square error function comprises a summation of weighted errors.

14. The system of claim 13, wherein the predictive model optimizes the weights using training data collected from a population of subjects, wherein the optimizing comprises identifying weights that minimize each subject's loss function.

15. The system of claim 14, wherein the optimizing comprises for each subject randomly selecting a subset of candidates from the population.

16. The system of claim 15, wherein the optimizing comprises for each candidate setting a second weight corresponding to the second glucose level classification to one.

17. The system of claim 16, wherein the optimizing comprises for each candidate setting a first weight and a third weight corresponding to the first glucose level classification and the third glucose level classification to values within a range.

18. The system of claim 17, wherein the optimizing comprises for each candidate deriving a fitness value by applying the predictive model to the corresponding training data of the subset of candidates, wherein the fitness value comprises output of the loss function.

19. The system of claim 18, wherein the optimizing comprises adjusting the first weight and the third weight of candidates and candidate offspring in the subset using a genetic algorithm and information of candidate and candidate offspring fitness values.

20. The system of claim 19, wherein the optimizing comprises for each subject iteratively applying the greedy algorithm across a plurality of generations to identify optimum weights for the subject.
